SEQUENCE LISTING (20 sequences)

SEQ ID NO: 1 - DNA, 6408 bp, Homo sapiens
  Plexin-B1, human, cDNA (coding sequence)
atgcctgctctgggcccagctcttctccaggctctctgggccgggtgggtcctcaccctccagccccttccaccaactgcattcactcccaatggcacgtatctgcagcacctggcaagggaccccacctcaggcaccctctacctgggggctaccaacttcctgttccagctgagccctgggctgcagctggaggccacagtgtccaccggccctgtgctagacagcagggactgcctgccacctgtgatgcctgatgagtgcccccaggcccagcctaccaacaacccgaatcagctgctcctggtgagcccaggggccctggtggtatgcgggagcgtgcaccagggggtctgtgaacagcggcgcctggggcagctcgagcagctgctgctgcggccagagcggcctggggacacacaatatgtggctgccaatgatcctgcggtcagcacggtggggctggtagcccagggcttggcaggggagcccctcctgtttgtggggcgaggatacaccagcaggggtgtggggggtggcattccacccatcacaacccgggccctgtggccgcccgacccccaagctgccttctcctatgaggagacagccaagctggcagtgggccgcctctccgagtacagccaccacttcgtgagtgcctttgcacgtggggccagcgcctacttcctgttcctgcggcgggacctgcaggctcagtctagagcttttcgtgcctatgtatctcgagtgtgtctccgggaccagcactactactcctatgtggagttgcctctggcctgcgaaggtggccgctacgggctgatccaggctgcagctgtggccacgtccagggaggtggcgcatggggaggtgctctttgcagctttctcctcggctgcaccccccactgtgggccggcccccatcggcggctgctggggcatctggagcctctgccctctgtgccttccccctggatgaggtggaccggcttgctaatcgcacgcgagatgcctgctacacccgggagggtcgtgctgaggatgggaccgaggtggcctacatcgagtatgatgtcaattctgactgtgcacagctgccagtggacaccctggatgcttatccctgtggctcagaccacacgcccagccccatggccagccgggtcccgctggaagccacaccaattctggagtggccagggattcagctaacagctgtggcagtcaccatggaagatggacacaccatcgctttcctgggtgatagtcaagggcagctgcacagggtctacttgggcccagggagcgatggccacccatactccacacagagcatccagcaggggtctgcagtgagcagagacctcacctttgatgggacctttgagcacctgtatgtcatgacccagagcacacttctgaaggttcctgtggcttcctgtgctcagcacctggactgtgcatcttgccttgctcacagggacccatactgtgggtggtgcgtgctccttggcaggtgcagtcgccgttctgagtgctcgaggggccagggcccagagcagtggctatggagcttccagcctgagctgggctgtctgcaagtggcagccatgagtcctgccaacatcagccgagaggagacgagggaggttttcctatcagtgccagacctgccacccctgtggccaggggagtcatattcctgccactttggggaacatcagagtcctgccctgctgactggttctggtgtgatgtgcccctccccagaccctagtgaggccccagtgctgccgagaggagccgactacgtatccgtgagcgtggagctcagatttggcgctgttgtgatcgccaaaacttccctctctttctatgactgtgtggcggtcactgaactccgcccatctgcgcagtgccaggcctgtgtgagcagccgctgggggtgtaactggtgtgtctggcagcacctgtgcacccacaaggcctcgtgtgatgctgggcccatggttgcaagccatcagagcccgcttgtctccccagaccctcctgcaagaggtggacccagcccctccccacccacagcccccaaagccctggccacccctgctcctgacacccttcccgtggagcctggggctccctccacagccacagcttcggacatctcacctggggctagtccttccctgctcagcccctgggggccatgggcaggttctggctccatatcttcccctggctccacagggtcgcctctccatgaggagccctcccctcccagcccccaaaatggacctggaaccgctgtccctgcccccactgacttcagaccctcagccacacctgaggacctcttggcctccccgctgtcaccctcagaggtagcagcagtgccccctgcagaccctggccccgaggctcttcatcccacagtgcccctggacctgccccctgccactgttcctgccaccactttcccaggggccatgggctccgtgaagcccgccctggactggctcacgagagaaggcggcgagctgcccgaggcggacgagtggacggggggtgacgcacccgccttctccacttccaccctcctctcaggtgatggagactcagcagagcttgagggccctcccgcccccctcatcctcccgtccagcctcgactaccagtatgacacccccgggctctgggagctggaagaggcgaccttgggggcaagctcctgcccctgtgtggagagcgttcagggctccacgttgatgccggtccatgtggagcgggaaatccggctgctaggcaggaacctgcaccttttccaggatggcccaggagacaatgagtgtgtgatggagctggagggcctcgaggtggtggttgaggcccgggtcgagtgtgagccacctccagatacccagtgccatgtcacctgccagcagcaccagctcagctatgaggctctgcagccggagctccgtgtggggctgtttctgcgtcgggccggccgtctgcgtgtggacagtgctgaggggctgcatgtggtactgtatgactgttccgtgggacatggagactgcagccgctgccaaactgccatgccccagtatggctgtgtgtggtgtgagggggagcgtccacgttgtgtgacccgggaggcctgtggtgaggctgaggctgtggccacccagtgcccagcgcccctcatccactcggtggagccactgactgggcctgtagacggaggcacccgtgtcaccatcaggggctccaacctgggccagcatgtgcaggatgtgctgggcatggtcacggtggctggagtgccctgtgctgtggatgcccaggagtacgaggtctccagcagcctcgtgtgcatcaccggggccagtggggaggaggtggccggcgccacagcggtggaggtgccgggaagaggacgtggtgtctcagaacacgactttgcctaccaggatccgaaggtccattccatcttcccggcccgcggccccagagctgggggcacccgtctcaccctgaatggctccaagctcctgactgggcggctggaggacatccgagtggtggttggagaccagccttgtcacttgctgccggagcagcagtcagaacaactgcggtgtgagaccagcccacgccccacgcctgccacgctccctgtggctgtgtggtttggggccacggagcggaggcttcaacgcggacagttcaagtataccttggaccccaacatcacctctgctggccccaccaagagcttcctcagtggaggacgtgagatatgcgtccgtggccagaatctggacgtggtacagacgccaagaatccgggtgaccgtggtctcgagaatgctgcagcccagccaggggcttggacggaggcgtcgcgtggtcccggagacggcatgttcccttggaccctcctgcagtagccagcaatttgaggagccgtgccatgtcaactcctcccagctcatcacgtgccgcacacctgccctcccaggcctgcctgaggacccctgggtccgggtggaatttatccttgacaacctggtctttgactttgcaacactgaaccccacacctttctcctatgaggccgaccccaccctgcagccactcaaccctgaggaccccaccatgccattccggcacaagcctgggagtgtgttctccgtggagggggagaacctggaccttgcaatgtccaaggaggaggtggtggctatgataggggatggcccctgtgtggtgaagacgctgacgcggcaccacctgtactgcgagccccccgtggagcagcccctgccacggcaccatgccctccgagaggcacctgactctttgcctgagttcacggtgcagatggggaacttgcgcttctccctgggtcacgtgcagtatgacggcgagagccctggggcttttcctgtggcagcccaggtgggcttgggggtgggcacctctcttctggctctgggtgtcatcatcattgtcctcatgtacaggaggaagagcaagcaggccctgagggactataagaaggttcagatccagctggagaatctggagagcagtgtgcgggaccgctgcaagaaggaattcacagacctcatgactgagatgaccgatctcaccagtgacctcctgggcagcggcatccccttcctcgactacaaggtgtatgcggagaggatcttcttccctgggcaccgcgagtcgcccttgcaccgggacctgggtgtgcctgagagcagacggcccactgtggagcaagggctggggcagctctctaacctgctcaacagcaagctcttcctcaccaagttcatccacacgctggagagccagcgcaccttttcagctcgggaccgtgcctacgtggcatctctgctcaccgtggcactgcatgggaagcttgagtatttcactgacatcctccgcactctgctcagtgacctggttgcccagtatgtggccaagaaccccaagctgatgctgcgcaggacagagactgtggtggagaagctgctcaccaactggatgtccatctgtctgtataccttcgtgagggactccgtaggggagcctctgtacatgctctttcgagggattaagcaccaagtggataaggggccagtggacagtgtgacaggcaaggccaaatacaccttgaacgacaaccgcctgctcagagaggatgtggagtaccgtcccctgaccttgaatgcactattggctgtggggcctggggcaggagaggcccagggcgtgcccgtgaaggtcctagactgtgacaccatctcccaggcaaaggagaagatgctggaccagctttataaaggagtgcctctcacccagcggccagaccctcgcacccttgatgttgagtggcggtctggggtggccgggcacctcattctttctgacgaggatgtcacttctgaggtccagggtctgtggaggcgcctgaacacactgcagcattacaaggtcccagatggagcaactgtggccctcgtcccctgcctcaccaagcatgtgctccgggaaaaccaggattatgtccctggagagcggaccccaatgctggaggatgtagatgaggggggcatccggccctggcacctggtgaagccaagtgatgagccggagccgcccaggcctcggaggggcagccttcggggcggggagcgtgagcgcgccaaggccatccctgagatctacctgacccgcctgctgtccatgaagggcaccctgcagaagttcgtggatgacctgttccaggtgattctcagcaccagccgccccgtgccgctcgctgtgaagtacttctttgacctgctggatgagcaggcccagcagcatggcatctccgaccaggacaccatccacatctggaagaccaacagcttgcctctgaggttctggatcaatataataaaaaacccgcagtttgtgttcgacgtgcaaacatctgataacatggatgcggtgctccttgtcattgcacagaccttcatggacgcctgcaccctggccgaccacaagctgggccgggactccccgatcaacaaacttctgtatgcacgggacattccccggtacaagcggatggtggaaaggtactatgcagacatcagacagactgtcccagccagcgaccaagagatgaactctgtcctggctgaactgtcctggaactactccggagacctcggggcgcgagtggccctgcatgaactctacaagtacatcaacaagtactatgaccagatcatcactgccctggaggaggatggcacggcccagaagatgcagctgggctatcggctccagcagattgcagctgctgtggaaaacaaggtcacagatctatag

SEQ ID NO: 2 - DNA, 5517 bp, Homo sapiens
  Plexin-B2, human, cDNA (coding sequence)
atggcactgcagctctgggccctgaccctgctgggcctgctgggcgcaggtgccagcctgaggccccgcaagctggacttcttccgcagcgagaaagagctgaaccacctggctgtggatgaggcctcaggcgtggtgtacctgggggcggtgaatgccctctaccagctggatgcgaagctgcagctggagcagcaggtggccacgggcccggccctggacaacaagaagtgcacgccgcccatcgaggccagccagtgccatgaggctgagatgactgacaatgtcaaccagctgctgctgctcgaccctcccaggaagcgcctggtggagtgcggcagcctcttcaagggcatctgcgctctgcgcgccctgagcaacatctccctccgcctgttctacgaggacggcagcggggagaagtctttcgtggccagcaatgatgagggcgtggccacagtggggctggtgagctccacgggtcctggtggtgaccgcgtgctgtttgtgggcaaaggcaatgggccacacgacaacggcatcatcgtgagcactcggctgttggaccggactgacagcagggaggcctttgaagcctacacggaccacgccacctacaaggccggctacctgtccaccaacacacagcagttcgtggcggccttcgaggacggcccctacgtcttctttgtcttcaaccagcaggacaagcacccggcccggaaccgcacgctgctggcacgcatgtgcagagaagaccccaactactactcctacctggagatggacctgcagtgccgggaccccgacatccacgccgctgcctttggcacctgcctggccgcctccgtggctgcgcctggctctggcagggtgctatatgctgtcttcagcagagacagccggagcagtggggggcccggtgcgggcctctgcctgttcccgctggacaaggtgcacgccaagatggaggccaaccgcaacgcctgttacacaggcacccgggaggcccgtgacatcttctacaagcccttccacggcgatatccagtgcggcggccacgcgccgggctccagcaagagcttcccatgtggctcggagcacctgccctacccgctgggcagccgcgacgggctcagaggcacagccgtgctgcagcgtggaggcctgaacctcacggccgtgacggtcgccgccgagaacaaccacactgttgcttttctgggcacctctgatggccggatcctcaaggtgtacctcaccccagatggcacctcctcagagtacgactctatccttgtggagataaacaagagagtcaagcgcgacctggtactgtctggagacctgggcagcctgtacgccatgacccaggacaaggtgttccggctgccggtgcaggagtgcctgagctacccgacctgcacccagtgccgcgactcccaggacccctactgcggctggtgcgtcgtcgagggacgatgcacccggaaggccgagtgtccgcgggccgaggaggccagccactggctgtggagccgaagcaagtcctgcgtggccgtcaccagcgcccagccacagaacatgagccggcgggcccagggggaggtgcagctgaccgtcagccccctccctgccctgagcgaggaggacgagttgctgtgcctttttggggagtcgccgccacaccccgcccgcgtggagggcgaggccgtcatctgcaactccccaagcagcatccccgtcacaccgccaggccaggaccacgtggccgtgaccatccagctcctccttagacgaggcaacatcttcctcacgtcctaccagtaccccttctacgactgccgccaggccatgagcctggaggagaacctgccgtgcatctcctgcgtgagcaaccgctggacctgccagtgggacctgcgctaccacgagtgccgggaggcttcgcccaaccctgaggacggcatcgtccgtgcccacatggaggacagctgtccccagttcctgggacccagccccctggtgatccccatgaaccacgagacagatgtgaacttccagggcaagaacctggacaccgtgaagggttcctccctgcacgtgggcagtgacttgctcaagttcatggagccggtgaccatgcaggaatctgggaccttcgcctttcggaccccaaagctgtcccacgatgccaacgagacgctgcccctgcacctctacgtcaagtcttacggcaagaatatcgacagcaagctccatgtgaccctctacaactgctcctttggccgcagcgactgcagcctgtgccgggccgctaaccccgactacaggtgtgcgtggtgcgggggccagagcaggtgcgtgtatgaggccctgtgcaacaccacctccgagtgcccgccgcccgtcatcaccaggatccagcctgagacgggccccctgggtgggggcatccgcatcaccatcctggggtccaatttgggcgtccaagcaggggacatccagaggatctctgtggccggccggaactgctcctttcagccggaacgttactccgtgtccacccggatcgtgtgtgtgatcgaggctgcggagacgcctttcacggggggtgtcgaggtggacgtcttcgggaaactgggccgttcgcctcccaatgtccagttcaccttccaacagcccaagcctctcagtgtggagccgcagcagggaccgcaggcgggcggcaccacactgaccatccacggcacccacctggacacgggctcccaggaggacgtgcgggtgaccctcaacggcgtcccgtgtaaagtgacgaagtttggggcgcagctccagtgtgtcactggcccccaggcgacacggggccagatgcttctggaggtctcctacggggggtcccccgtgcccaaccccggcatcttcttcacctaccgcgaaaaccccgtactgcgagccttcgagccgctacgaagctttgccagtggtggccgcagcatcaacgtcacgggtcagggcttcagcctgatccagaggtttgccatggtggtcatcgcggagcccctgcagtcctggcagccgccgcgggaggctgaatccctgcagcccatgacggtggtgggtacagactacgtgttccacaatgacaccaaggtcgtcttcctgtccccggctgtgcctgaggagccagaggcctacaacctcacggtgctgatcgagatggacgggcaccgtgccctgctcagaacagaggccggggccttcgagtacgtgcctgaccccacctttgagaacttcacaggtggcgtcaagaagcaggtcaacaagctcatccacgcccggggcaccaatctgaacaaggcgatgacgctgcaggaggccgaggccttcgtgggtgccgagcgctgcaccatgaagacgctgacggagaccgacctgtactgtgagcccccggaggtgcagcccccgcccaagcggcggcagaaacgagacaccacacacaacctgcccgagttcattgtgaagttcggctctcgcgagtgggtgctgggccgcgtggagtacgacacacgggtgagcgacgtgccgctcagcctcatcttgccgctggtcatcgtgcccatggtggtcgtcatcgcggtgtctgtctactgctactggaggaagagccagcaggccgaacgagagtatgagaagatcaagtcccagctggagggcctggaggagagcgtgcgggaccgctgcaagaaggaattcacagacctgatgatcgagatggaggaccagaccaacgacgtgcacgaggccggcatccccgtgctggactacaagacctacaccgaccgcgtcttcttcctgccctccaaggacggcgacaaggacgtgatgatcaccggcaagctggacatccctgagccgcggcggccggtggtggagcaggccctctaccagttctccaacctgctgaacagcaagtctttcctcatcaatttcatccacaccctggagaaccagcgggagttctcggcccgcgccaaggtctacttcgcgtccctgctgacggtggcgctgcacgggaaactggagtactacacggacatcatgcacacgctcttcctggagctcctggagcagtacgtggtggccaagaaccccaagctgatgctgcgcaggtctgagactgtggtggagaggatgctgtccaactggatgtccatctgcctgtaccagtacctcaaggacagtgccggggagcccctgtacaagctcttcaaggccatcaaacatcaggtggaaaagggcccggtggatgcggtacagaagaaggccaagtacactctcaacgacacggggctgctgggggatgatgtggagtacgcacccctgacggtgagcgtgatcgtgcaggacgagggagtggacgccatcccggtgaaggtcctcaactgtgacaccatctcccaggtcaaggagaagatcattgaccaggtgtaccgtgggcagccctgctcctgctggcccaggccagacagcgtggtcctggagtggcgtccgggctccacagcgcagatcctgtcggacctggacctgacgtcacagcgggagggccggtggaagcgcgtcaacacccttatgcactacaatgtccgggatggagccaccctcatcctgtccaaggtgggggtctcccagcagccggaggacagccagcaggacctgcctggggagcgccatgccctcctggaggaggagaaccgggtgtggcacctggtgcggccgaccgacgaggtggacgagggcaagtccaagagaggcagcgtgaaagagaaggagcggacgaaggccatcaccgagatctacctgacgcggctgctctcagtcaagggcacactgcagcagtttgtggacaacttcttccagagcgtgctggcgcctgggcacgcggtgccacctgcagtcaagtacttcttcgacttcctggacgagcaggcagagaagcacaacatccaggatgaagacaccatccacatctggaagacgaacagcttaccgctccggttctgggtgaacatcctcaagaacccccacttcatctttgacgtgcatgtccacgaggtggtggacgcctcgctgtcagtcatcgcgcagaccttcatggatgcctgcacgcgcacggagcataagctgagccgcgattctcccagcaacaagctgctgtacgccaaggagatctccacctacaagaagatggtggaggattactacaaggggatccggcagatggtgcaggtcagcgaccaggacatgaacacacacctggcagagatttcccgggcgcacacggactccttgaacaccctcgtggcactccaccagctctaccaatacacgcagaagtactatgacgagatcatcaatgccttggaggaggatcctgccgcccagaagatgcagctggccttccgcctgcagcagattgccgctgcactggagaacaaggtcactgacctctga

SEQ ID NO: 3 - protein, 2135 aa, Homo sapiens
  Plexin-B1, human, Protein
MPALGPALLQALWAGWVLTLQPLPPTAFTPNGTYLQHLARDPTSGTLYLGATNFLFQLSPGLQLEATVSTGPVLDSRDCLPPVMPDECPQAQPTNNPNQLLLVSPGALVVCGSVHQGVCEQRRLGQLEQLLLRPERPGDTQYVAANDPAVSTVGLVAQGLAGEPLLFVGRGYTSRGVGGGIPPITTRALWPPDPQAAFSYEETAKLAVGRLSEYSHHFVSAFARGASAYFLFLRRDLQAQSRAFRAYVSRVCLRDQHYYSYVELPLACEGGRYGLIQAAAVATSREVAHGEVLFAAFSSAAPPTVGRPPSAAAGASGASALCAFPLDEVDRLANRTRDACYTREGRAEDGTEVAYIEYDVNSDCAQLPVDTLDAYPCGSDHTPSPMASRVPLEATPILEWPGIQLTAVAVTMEDGHTIAFLGDSQGQLHRVYLGPGSDGHPYSTQSIQQGSAVSRDLTFDGTFEHLYVMTQSTLLKVPVASCAQHLDCASCLAHRDPYCGWCVLLGRCSRRSECSRGQGPEQWLWSFQPELGCLQVAAMSPANISREETREVFLSVPDLPPLWPGESYSCHFGEHQSPALLTGSGVMCPSPDPSEAPVLPRGADYVSVSVELRFGAVVIAKTSLSFYDCVAVTELRPSAQCQACVSSRWGCNWCVWQHLCTHKASCDAGPMVASHQSPLVSPDPPARGGPSPSPPTAPKALATPAPDTLPVEPGAPSTATASDISPGASPSLLSPWGPWAGSGSISSPGSTGSPLHEEPSPPSPQNGPGTAVPAPTDFRPSATPEDLLASPLSPSEVAAVPPADPGPEALHPTVPLDLPPATVPATTFPGAMGSVKPALDWLTREGGELPEADEWTGGDAPAFSTSTLLSGDGDSAELEGPPAPLILPSSLDYQYDTPGLWELEEATLGASSCPCVESVQGSTLMPVHVEREIRLLGRNLHLFQDGPGDNECVMELEGLEVVVEARVECEPPPDTQCHVTCQQHQLSYEALQPELRVGLFLRRAGRLRVDSAEGLHVVLYDCSVGHGDCSRCQTAMPQYGCVWCEGERPRCVTREACGEAEAVATQCPAPLIHSVEPLTGPVDGGTRVTIRGSNLGQHVQDVLGMVTVAGVPCAVDAQEYEVSSSLVCITGASGEEVAGATAVEVPGRGRGVSEHDFAYQDPKVHSIFPARGPRAGGTRLTLNGSKLLTGRLEDIRVVVGDQPCHLLPEQQSEQLRCETSPRPTPATLPVAVWFGATERRLQRGQFKYTLDPNITSAGPTKSFLSGGREICVRGQNLDVVQTPRIRVTVVSRMLQPSQGLGRRRRVVPETACSLGPSCSSQQFEEPCHVNSSQLITCRTPALPGLPEDPWVRVEFILDNLVFDFATLNPTPFSYEADPTLQPLNPEDPTMPFRHKPGSVFSVEGENLDLAMSKEEVVAMIGDGPCVVKTLTRHHLYCEPPVEQPLPRHHALREAPDSLPEFTVQMGNLRFSLGHVQYDGESPGAFPVAAQVGLGVGTSLLALGVIIIVLMYRRKSKQALRDYKKVQIQLENLESSVRDRCKKEFTDLMTEMTDLTSDLLGSGIPFLDYKVYAERIFFPGHRESPLHRDLGVPESRRPTVEQGLGQLSNLLNSKLFLTKFIHTLESQRTFSARDRAYVASLLTVALHGKLEYFTDILRTLLSDLVAQYVAKNPKLMLRRTETVVEKLLTNWMSICLYTFVRDSVGEPLYMLFRGIKHQVDKGPVDSVTGKAKYTLNDNRLLREDVEYRPLTLNALLAVGPGAGEAQGVPVKVLDCDTISQAKEKMLDQLYKGVPLTQRPDPRTLDVEWRSGVAGHLILSDEDVTSEVQGLWRRLNTLQHYKVPDGATVALVPCLTKHVLRENQDYVPGERTPMLEDVDEGGIRPWHLVKPSDEPEPPRPRRGSLRGGERERAKAIPEIYLTRLLSMKGTLQKFVDDLFQVILSTSRPVPLAVKYFFDLLDEQAQQHGISDQDTIHIWKTNSLPLRFWINIIKNPQFVFDVQTSDNMDAVLLVIAQTFMDACTLADHKLGRDSPINKLLYARDIPRYKRMVERYYADIRQTVPASDQEMNSVLAELSWNYSGDLGARVALHELYKYINKYYDQIITALEEDGTAQKMQLGYRLQQIAAAVENKVTDL

SEQ ID NO: 4 - protein, 1838 aa, Homo sapiens
  Plexin-B2, human, Protein
MALQLWALTLLGLLGAGASLRPRKLDFFRSEKELNHLAVDEASGVVYLGAVNALYQLDAKLQLEQQVATGPALDNKKCTPPIEASQCHEAEMTDNVNQLLLLDPPRKRLVECGSLFKGICALRALSNISLRLFYEDGSGEKSFVASNDEGVATVGLVSSTGPGGDRVLFVGKGNGPHDNGIIVSTRLLDRTDSREAFEAYTDHATYKAGYLSTNTQQFVAAFEDGPYVFFVFNQQDKHPARNRTLLARMCREDPNYYSYLEMDLQCRDPDIHAAAFGTCLAASVAAPGSGRVLYAVFSRDSRSSGGPGAGLCLFPLDKVHAKMEANRNACYTGTREARDIFYKPFHGDIQCGGHAPGSSKSFPCGSEHLPYPLGSRDGLRGTAVLQRGGLNLTAVTVAAENNHTVAFLGTSDGRILKVYLTPDGTSSEYDSILVEINKRVKRDLVLSGDLGSLYAMTQDKVFRLPVQECLSYPTCTQCRDSQDPYCGWCVVEGRCTRKAECPRAEEASHWLWSRSKSCVAVTSAQPQNMSRRAQGEVQLTVSPLPALSEEDELLCLFGESPPHPARVEGEAVICNSPSSIPVTPPGQDHVAVTIQLLLRRGNIFLTSYQYPFYDCRQAMSLEENLPCISCVSNRWTCQWDLRYHECREASPNPEDGIVRAHMEDSCPQFLGPSPLVIPMNHETDVNFQGKNLDTVKGSSLHVGSDLLKFMEPVTMQESGTFAFRTPKLSHDANETLPLHLYVKSYGKNIDSKLHVTLYNCSFGRSDCSLCRAANPDYRCAWCGGQSRCVYEALCNTTSECPPPVITRIQPETGPLGGGIRITILGSNLGVQAGDIQRISVAGRNCSFQPERYSVSTRIVCVIEAAETPFTGGVEVDVFGKLGRSPPNVQFTFQQPKPLSVEPQQGPQAGGTTLTIHGTHLDTGSQEDVRVTLNGVPCKVTKFGAQLQCVTGPQATRGQMLLEVSYGGSPVPNPGIFFTYRENPVLRAFEPLRSFASGGRSINVTGQGFSLIQRFAMVVIAEPLQSWQPPREAESLQPMTVVGTDYVFHNDTKVVFLSPAVPEEPEAYNLTVLIEMDGHRALLRTEAGAFEYVPDPTFENFTGGVKKQVNKLIHARGTNLNKAMTLQEAEAFVGAERCTMKTLTETDLYCEPPEVQPPPKRRQKRDTTHNLPEFIVKFGSREWVLGRVEYDTRVSDVPLSLILPLVIVPMVVVIAVSVYCYWRKSQQAEREYEKIKSQLEGLEESVRDRCKKEFTDLMIEMEDQTNDVHEAGIPVLDYKTYTDRVFFLPSKDGDKDVMITGKLDIPEPRRPVVEQALYQFSNLLNSKSFLINFIHTLENQREFSARAKVYFASLLTVALHGKLEYYTDIMHTLFLELLEQYVVAKNPKLMLRRSETVVERMLSNWMSICLYQYLKDSAGEPLYKLFKAIKHQVEKGPVDAVQKKAKYTLNDTGLLGDDVEYAPLTVSVIVQDEGVDAIPVKVLNCDTISQVKEKIIDQVYRGQPCSCWPRPDSVVLEWRPGSTAQILSDLDLTSQREGRWKRVNTLMHYNVRDGATLILSKVGVSQQPEDSQQDLPGERHALLEEENRVWHLVRPTDEVDEGKSKRGSVKEKERTKAITEIYLTRLLSVKGTLQQFVDNFFQSVLAPGHAVPPAVKYFFDFLDEQAEKHNIQDEDTIHIWKTNSLPLRFWVNILKNPHFIFDVHVHEVVDASLSVIAQTFMDACTRTEHKLSRDSPSNKLLYAKEISTYKKMVEDYYKGIRQMVQVSDQDMNTHLAEISRAHTDSLNTLVALHQLYQYTQKYYDEIINALEEDPAAQKMQLAFRLQQIAAALENKVTDL

SEQ ID NO: 5 - DNA, 5859 bp, Homo sapiens
  PLXNB1-204 cDNA (coding sequence)
atgcctgctctgggcccagctcttctccaggctctctgggccgggtgggtcctcaccctccagccccttccaccaactgcattcactcccaatggcacgtatctgcagcacctggcaagggaccccacctcaggcaccctctacctgggggctaccaacttcctgttccagctgagccctgggctgcagctggaggccacagtgtccaccggccctgtgctagacagcagggactgcctgccacctgtgatgcctgatgagtgcccccaggcccagcctaccaacaacccgaatcagctgctcctggtgagcccaggggccctggtggtatgcgggagcgtgcaccagggggtctgtgaacagcggcgcctggggcagctcgagcagctgctgctgcggccagagcggcctggggacacacaatatgtggctgccaatgatcctgcggtcagcacggtggggctggtagcccagggcttggcaggggagcccctcctgtttgtggggcgaggatacaccagcaggggtgtggggggtggcattccacccatcacaacccgggccctgtggccgcccgacccccaagctgccttctcctatgaggagacagccaagctggcagtgggccgcctctccgagtacagccaccacttcgtgagtgcctttgcacgtggggccagcgcctacttcctgttcctgcggcgggacctgcaggctcagtctagagcttttcgtgcctatgtatctcgagtgtgtctccgggaccagcactactactcctatgtggagttgcctctggcctgcgaaggtggccgctacgggctgatccaggctgcagctgtggccacgtccagggaggtggcgcatggggaggtgctctttgcagctttctcctcggctgcaccccccactgtgggccggcccccatcggcggctgctggggcatctggagcctctgccctctgtgccttccccctggatgaggtggaccggcttgctaatcgcacgcgagatgcctgctacacccgggagggtcgtgctgaggatgggaccgaggtggcctacatcgagtatgatgtcaattctgactgtgcacagctgccagtggacaccctggatgcttatccctgtggctcagaccacacgcccagccccatggccagccgggtcccgctggaagccacaccaattctggagtggccagggattcagctaacagctgtggcagtcaccatggaagatggacacaccatcgctttcctgggtgatagtcaagggcagctgcacagggtctacttgggcccagggagcgatggccacccatactccacacagagcatccagcaggggtctgcagtgagcagagacctcacctttgatgggacctttgagcacctgtatgtcatgacccagagcacacttctgaaggttcctgtggcttcctgtgctcagcacctggactgtgcatcttgccttgctcacagggacccatactgtgggtggtgcgtgctccttggcaggtgcagtcgccgttctgagtgctcgaggggccagggcccagagcagtggctatggagcttccagcctgagctgggctgtctgcaagtggcagccatgagtcctgccaacatcagccgagaggagacgagggaggttttcctatcagtgccagacctgccacccctgtggccaggggagtcatattcctgccactttggggaacatcagagtcctgccctgctgactggttctggtgtgatgtgcccctccccagaccctagtgaggccccagtgctgccgagaggagccgactacgtatccgtgagcgtggagctcagatttggcgctgttgtgatcgccaaaacttccctctctttctatgactgtgtggcggtcactgaactccgcccatctgcgcagtgccaggcctgtgtgagcagccgctgggggtgtaactggtgtgtctggcagcacctgtgcacccacaaggcctcgtgtgatgctgggcccatggttgcaagccatcagagcccgcttgtctccccagaccctcctgcaagaggtgatggagactcagcagagcttgagggccctcccgcccccctcatcctcccgtccagcctcgactaccagtatgacacccccgggctctgggagctggaagaggcgaccttgggggcaagctcctgcccctgtgtggagagcgttcagggctccacgttgatgccggtccatgtggagcgggaaatccggctgctaggcaggaacctgcaccttttccaggatggcccaggagacaatgagtgtgtgatggagctggagggcctcgaggtggtggttgaggcccgggtcgagtgtgagccacctccagatacccagtgccatgtcacctgccagcagcaccagctcagctatgaggctctgcagccggagctccgtgtggggctgtttctgcgtcgggccggccgtctgcgtgtggacagtgctgaggggctgcatgtggtactgtatgactgttccgtgggacatggagactgcagccgctgccaaactgccatgccccagtatggctgtgtgtggtgtgagggggagcgtccacgttgtgtgacccgggaggcctgtggtgaggctgaggctgtggccacccagtgcccagcgcccctcatccactcggtggagccactgactgggcctgtagacggaggcacccgtgtcaccatcaggggctccaacctgggccagcatgtgcaggatgtgctgggcatggtcacggtggctggagtgccctgtgctgtggatgcccaggagtacgaggtctccagcagcctcgtgtgcatcaccggggccagtggggaggaggtggccggcgccacagcggtggaggtgccgggaagaggacgtggtgtctcagaacacgactttgcctaccaggatccgaaggtccattccatcttcccggcccgcggccccagagctgggggcacccgtctcaccctgaatggctccaagctcctgactgggcggctggaggacatccgagtggtggttggagaccagccttgtcacttgctgccggagcagcagtcagaacaactgcggtgtgagaccagcccacgccccacgcctgccacgctccctgtggctgtgtggtttggggccacggagcggaggcttcaacgcggacagttcaagtataccttggaccccaacatcacctctgctggccccaccaagagcttcctcagtggaggacgtgagatatgcgtccgtggccagaatctggacgtggtacagacgccaagaatccgggtgaccgtggtctcgagaatgctgcagcccagccaggggcttggacggaggcgtcgcgtggtcccggagacggcatgttcccttggaccctcctgcagtagccagcaatttgaggagccgtgccatgtcaactcctcccagctcatcacgtgccgcacacctgccctcccaggcctgcctgaggacccctgggtccgggtggaatttatccttgacaacctggtctttgactttgcaacactgaaccccacacctttctcctatgaggccgaccccaccctgcagccactcaaccctgaggaccccaccatgccattccggcacaagcctgggagtgtgttctccgtggagggggagaacctggaccttgcaatgtccaaggaggaggtggtggctatgataggggatggcccctgtgtggtgaagacgctgacgcggcaccacctgtactgcgagccccccgtggagcagcccctgccacggcaccatgccctccgagaggcacctgactctttgcctgagttcacggtgcagatggggaacttgcgcttctccctgggtcacgtgcagtatgacggcgagagccctggggcttttcctgtggcagcccaggtgggcttgggggtgggcacctctcttctggctctgggtgtcatcatcattgtcctcatgtacaggaggaagagcaagcaggccctgagggactataagaaggttcagatccagctggagaatctggagagcagtgtgcgggaccgctgcaagaaggaattcacagacctcatgactgagatgaccgatctcaccagtgacctcctgggcagcggcatccccttcctcgactacaaggtgtatgcggagaggatcttcttccctgggcaccgcgagtcgcccttgcaccgggacctgggtgtgcctgagagcagacggcccactgtggagcaagggctggggcagctctctaacctgctcaacagcaagctcttcctcaccaagttcatccacacgctggagagccagcgcaccttttcagctcgggaccgtgcctacgtggcatctctgctcaccgtggcactgcatgggaagcttgagtatttcactgacatcctccgcactctgctcagtgacctggttgcccagtatgtggccaagaaccccaagctgatgctgcgcaggacagagactgtggtggagaagctgctcaccaactggatgtccatctgtctgtataccttcgtgagggactccgtaggggagcctctgtacatgctctttcgagggattaagcaccaagtggataaggggccagtggacagtgtgacaggcaaggccaaatacaccttgaacgacaaccgcctgctcagagaggatgtggagtaccgtcccctgaccttgaatgcactattggctgtggggcctggggcaggagaggcccagggcgtgcccgtgaaggtcctagactgtgacaccatctcccaggcaaaggagaagatgctggaccagctttataaaggagtgcctctcacccagcggccagaccctcgcacccttgatgttgagtggcggtctggggtggccgggcacctcattctttctgacgaggatgtcacttctgaggtccagggtctgtggaggcgcctgaacacactgcagcattacaaggtcccagatggagcaactgtggccctcgtcccctgcctcaccaagcatgtgctccgggaaaaccaggattatgtccctggagagcggaccccaatgctggaggatgtagatgaggggggcatccggccctggcacctggtgaagccaagtgatgagccggagccgcccaggcctcggaggggcagccttcggggcggggagcgtgagcgcgccaaggccatccctgagatctacctgacccgcctgctgtccatgaagggcaccctgcagaagttcgtggatgacctgttccaggtgattctcagcaccagccgccccgtgccgctcgctgtgaagtacttctttgacctgctggatgagcaggcccagcagcatggcatctccgaccaggacaccatccacatctggaagaccaacagcttgcctctgaggttctggatcaatataataaaaaacccgcagtttgtgttcgacgtgcaaacatctgataacatggatgcggtgctccttgtcattgcacagaccttcatggacgcctgcaccctggccgaccacaagctgggccgggactccccgatcaacaaacttctgtatgcacgggacattccccggtacaagcggatggtggaaaggtactatgcagacatcagacagactgtcccagccagcgaccaagagatgaactctgtcctggctgaactgtcctggaactactccggagacctcggggcgcgagtggccctgcatgaactctacaagtacatcaacaagtactatgaccagatcatcactgccctggaggaggatggcacggcccagaagatgcagctgggctatcggctccagcagattgcagctgctgtggaaaacaaggtcacagatctatag

SEQ ID NO: 6 - protein, 1952 aa, Homo sapiens
  PLXNB1-204 protein
MPALGPALLQALWAGWVLTLQPLPPTAFTPNGTYLQHLARDPTSGTLYLGATNFLFQLSPGLQLEATVSTGPVLDSRDCLPPVMPDECPQAQPTNNPNQLLLVSPGALVVCGSVHQGVCEQRRLGQLEQLLLRPERPGDTQYVAANDPAVSTVGLVAQGLAGEPLLFVGRGYTSRGVGGGIPPITTRALWPPDPQAAFSYEETAKLAVGRLSEYSHHFVSAFARGASAYFLFLRRDLQAQSRAFRAYVSRVCLRDQHYYSYVELPLACEGGRYGLIQAAAVATSREVAHGEVLFAAFSSAAPPTVGRPPSAAAGASGASALCAFPLDEVDRLANRTRDACYTREGRAEDGTEVAYIEYDVNSDCAQLPVDTLDAYPCGSDHTPSPMASRVPLEATPILEWPGIQLTAVAVTMEDGHTIAFLGDSQGQLHRVYLGPGSDGHPYSTQSIQQGSAVSRDLTFDGTFEHLYVMTQSTLLKVPVASCAQHLDCASCLAHRDPYCGWCVLLGRCSRRSECSRGQGPEQWLWSFQPELGCLQVAAMSPANISREETREVFLSVPDLPPLWPGESYSCHFGEHQSPALLTGSGVMCPSPDPSEAPVLPRGADYVSVSVELRFGAVVIAKTSLSFYDCVAVTELRPSAQCQACVSSRWGCNWCVWQHLCTHKASCDAGPMVASHQSPLVSPDPPARGDGDSAELEGPPAPLILPSSLDYQYDTPGLWELEEATLGASSCPCVESVQGSTLMPVHVEREIRLLGRNLHLFQDGPGDNECVMELEGLEVVVEARVECEPPPDTQCHVTCQQHQLSYEALQPELRVGLFLRRAGRLRVDSAEGLHVVLYDCSVGHGDCSRCQTAMPQYGCVWCEGERPRCVTREACGEAEAVATQCPAPLIHSVEPLTGPVDGGTRVTIRGSNLGQHVQDVLGMVTVAGVPCAVDAQEYEVSSSLVCITGASGEEVAGATAVEVPGRGRGVSEHDFAYQDPKVHSIFPARGPRAGGTRLTLNGSKLLTGRLEDIRVVVGDQPCHLLPEQQSEQLRCETSPRPTPATLPVAVWFGATERRLQRGQFKYTLDPNITSAGPTKSFLSGGREICVRGQNLDVVQTPRIRVTVVSRMLQPSQGLGRRRRVVPETACSLGPSCSSQQFEEPCHVNSSQLITCRTPALPGLPEDPWVRVEFILDNLVFDFATLNPTPFSYEADPTLQPLNPEDPTMPFRHKPGSVFSVEGENLDLAMSKEEVVAMIGDGPCVVKTLTRHHLYCEPPVEQPLPRHHALREAPDSLPEFTVQMGNLRFSLGHVQYDGESPGAFPVAAQVGLGVGTSLLALGVIIIVLMYRRKSKQALRDYKKVQIQLENLESSVRDRCKKEFTDLMTEMTDLTSDLLGSGIPFLDYKVYAERIFFPGHRESPLHRDLGVPESRRPTVEQGLGQLSNLLNSKLFLTKFIHTLESQRTFSARDRAYVASLLTVALHGKLEYFTDILRTLLSDLVAQYVAKNPKLMLRRTETVVEKLLTNWMSICLYTFVRDSVGEPLYMLFRGIKHQVDKGPVDSVTGKAKYTLNDNRLLREDVEYRPLTLNALLAVGPGAGEAQGVPVKVLDCDTISQAKEKMLDQLYKGVPLTQRPDPRTLDVEWRSGVAGHLILSDEDVTSEVQGLWRRLNTLQHYKVPDGATVALVPCLTKHVLRENQDYVPGERTPMLEDVDEGGIRPWHLVKPSDEPEPPRPRRGSLRGGERERAKAIPEIYLTRLLSMKGTLQKFVDDLFQVILSTSRPVPLAVKYFFDLLDEQAQQHGISDQDTIHIWKTNSLPLRFWINIIKNPQFVFDVQTSDNMDAVLLVIAQTFMDACTLADHKLGRDSPINKLLYARDIPRYKRMVERYYADIRQTVPASDQEMNSVLAELSWNYSGDLGARVALHELYKYINKYYDQIITALEEDGTAQKMQLGYRLQQIAAAVENKVTDL

SEQ ID NO: 7 - DNA, 704 bp, Homo sapiens
  PLXNB2-202 cDNA (coding sequence)
tacaagcccttccacggcgatatccagtgcggcggccacgcgccgggctccagcaagagcttcccatgtggctcggagcacctgccctacccgctgggcagccgcgacgggctcagaggcacagccgtgctgcagcgtggaggcctgaacctcacggccgtgacggtcgccgccgagaacaaccacactgttgcttttctggagtggcgtccgggctccacagcgcagatcctgtcggacctggacctgacgtcacagcgggagggccggtggaagcgcgtcaacacccttatgcactacaatgtccgggatggagccaccctcatcctgtccaaggtgggggtctcccagcagccggaggacagccagcaggacctgcctggggagcgccatgccctcctggaggaggagaaccgggtgtggcacctggtgcggccgaccgacgaggtggacgagggcaagtccaagagaggcagcgtgaaagagaaggagcggacgaaggccatcaccgagatctacctgacgcggctgctctcagtcaagggcacactgcagcagtttgtggacaacttcttccagagcgtgctggcgcctgggcacgcggtgccacctgcagtcaagtacttcttcgacttcctggacgagcaggcagagaagcacaacatccaggatgaagacaccatccacatctggaagacgaacag

SEQ ID NO: 8 - protein, 234 aa, Homo sapiens
  PLXNB2-202 protein
YKPFHGDIQCGGHAPGSSKSFPCGSEHLPYPLGSRDGLRGTAVLQRGGLNLTAVTVAAENNHTVAFLEWRPGSTAQILSDLDLTSQREGRWKRVNTLMHYNVRDGATLILSKVGVSQQPEDSQQDLPGERHALLEEENRVWHLVRPTDEVDEGKSKRGSVKEKERTKAITEIYLTRLLSVKGTLQQFVDNFFQSVLAPGHAVPPAVKYFFDFLDEQAEKHNIQDEDTIHIWKTN

SEQ ID NO: 9 - DNA, 485 bp, Homo sapiens
  PLXNB2-203 cDNA (coding sequence)
atggcactgcagctctgggccctgaccctgctgggcctgctgggcgcaggtgccagcctgaggccccgcaagctggacttcttccgcagcgagaaagagctgaaccacctggctgtggatgaggcctcaggcgtggtgtacctgggggcggtgaatgccctctaccagctggatgcgaagctgcagctggagcagcaggtggccacgggcccggccctggacaacaagaagtgcacgccgcccatcgaggccagccagtgccatgaggctgagatgactgacaatgtcaaccagctgctgctgctcgaccctcccaggaagcgcctggtggagtgcggcagcctcttcaagggcatctgcgctctgcgcgccctgagcaacatctccctccgcctgttctacgaggacggcagcggggagaagtctttcgtggccagcaatgatgagggcgtggccacagtggggctggtgagctccacgggtcc

SEQ ID NO: 10 - protein, 161 aa, Homo sapiens
  PLXNB2-203 protein
MALQLWALTLLGLLGAGASLRPRKLDFFRSEKELNHLAVDEASGVVYLGAVNALYQLDAKLQLEQQVATGPALDNKKCTPPIEASQCHEAEMTDNVNQLLLLDPPRKRLVECGSLFKGICALRALSNISLRLFYEDGSGEKSFVASNDEGVATVGLVSSTG

SEQ ID NO: 11 - DNA, 568 bp, Homo sapiens
  PLXNB2-204 cDNA (coding sequence)
  variation 1: /replace=“a,c,g,t”
nacgaagtttggggcgcagctccagtgtgtcactggcccccaggcgacacggggccagatgcttctggaggtctcctacggggggtcccccgtgcccaaccccggcatcttcttcacctaccgcgaaaaccccgtactgcgagccttcgagccgctacgaagctttgccaggtggtgggtacagactacgtgttccacaatgacaccaaggtcgtcttcctgtccccggctgtgcctgaggagccagaggcctacaacctcacggtgctgatcgagatggacgggcaccgtgccctgctcagaacagaggccggggccttcgagtacgtgcctgaccccacctttgagaacttcacaggtggcgtcaagaagcaggtcaacaagctcatccacgcccggggcaccaatctgaacaaggcgatgacgctgcaggaggccgaggccttcgtgggtgccgagcgctgcaccatgaagacgctgacggagaccgacctgtactgtgagcccccggaggtgcagcccccgcccaagcggcggcagaaacgagacaccacacacaacctgcccg

SEQ ID NO: 12 - protein, 189 aa, Homo sapiens
  PLXNB2-204 protein
  VARIANT 1: Ala,Cys,Asp,Glu,Phe,Gly,His,Ile,Lys,Leu,Met, Asn,Pro,Gln,Arg,Ser,Thr,Val,Trp,Tyr
XEVWGAAPVCHWPPGDTGPDASGGLLRGVPRAQPRHLLHLPRKPRTASLRAATKLCQVVGTDYVFHNDTKVVFLSPAVPEEPEAYNLTVLIEMDGHRALLRTEAGAFEYVPDPTFENFTGGVKKQVNKLIHARGTNLNKAMTLQEAEAFVGAERCTMKTLTETDLYCEPPEVQPPPKRRQKRDTTHNLP

SEQ ID NO: 13 - DNA, 1431 bp, Homo sapiens
  PLXNB2-205 cDNA (coding sequence
atggcactgcagctctgggccctgaccctgctgggcctgctgggcgcaggtgccagcctgaggccccgcaagctggacttcttccgcagcgagaaagagctgaaccacctggctgtggatgaggcctcaggcgtggtgtacctgggggcggtgaatgccctctaccagctggatgcgaagctgcagctggagcagcaggtggccacgggcccggccctggacaacaagaagtgcacgccgcccatcgaggccagccagtgccatgaggctgagatgactgacaatgtcaaccagctgctgctgctcgaccctcccaggaagcgcctggtggagtgcggcagcctcttcaagggcatctgcgctctgcgcgccctgagcaacatctccctccgcctgttctacgaggacggcagcggggagaagtctttcgtggccagcaatgatgagggcgtggccacagtggggctggtgagctccacgggtcctggtggtgaccgcgtgctgtttgtgggcaaaggcaatgggccacacgacaacggcatcatcgtgagcactcggctgttggaccggactgacagcagggaggcctttgaagcctacacggaccacgccacctacaaggccggctacctgtccaccaacacacagcagttcgtggcggccttcgaggacggcccctacgtcttctttgtcttcaaccagcaggacaagcacccggcccggaaccgcacgctgctggcacgcatgtgcagagaagaccccaactactactcctacctggagatggacctgcagtgccgggaccccgacatccacgccgctgcctttggcacctgcctggccgcctccgtggctgcgcctggctctggcagggtgctatatgctgtcttcagcagagacagccggagcagtggggggcccggtgcgggcctctgcctgttcccgctggacaaggtgcacgccaagatggaggccaaccgcaacgcctgttacacaggcacccgggaggcccgtgacatcttctacaagcccttccacggcgatatccagtgcggcggccacgcgccgggctccagcaagagcttcccatgtggctcggagcacctgccctacccgctgggcagccgcgacgggctcagaggcacagccgtgctgcagcgtggaggcctgaacctcacggccgtgacggtcgccgccgagaacaaccacactgttgcttttctgggcacctctgatggccggatcctcaaggtgtacctcaccccagatggcacctcctcagagtacgactctatccttgtggagataaacaagagagtcaagcgcgacctggtactgtctggagacctgggcagcctgtacgccatgacccaggacaaggtgttccggctgccggtgcaggagtgcctgagctacccgacctgcacccag

SEQ ID NO: 14 - protein, 477 aa, Homo sapiens
  PLXNB2-205 protein
MALQLWALTLLGLLGAGASLRPRKLDFFRSEKELNHLAVDEASGVVYLGAVNALYQLDAKLQLEQQVATGPALDNKKCTPPIEASQCHEAEMTDNVNQLLLLDPPRKRLVECGSLFKGICALRALSNISLRLFYEDGSGEKSFVASNDEGVATVGLVSSTGPGGDRVLFVGKGNGPHDNGIIVSTRLLDRTDSREAFEAYTDHATYKAGYLSTNTQQFVAAFEDGPYVFFVFNQQDKHPARNRTLLARMCREDPNYYSYLEMDLQCRDPDIHAAAFGTCLAASVAAPGSGRVLYAVFSRDSRSSGGPGAGLCLFPLDKVHAKMEANRNACYTGTREARDIFYKPFHGDIQCGGHAPGSSKSFPCGSEHLPYPLGSRDGLRGTAVLQRGGLNLTAVTVAAENNHTVAFLGTSDGRILKVYLTPDGTSSEYDSILVEINKRVKRDLVLSGDLGSLYAMTQDKVFRLPVQECLSYPTCTQ

SEQ ID NO: 15 - DNA, 254 bp, Homo sapiens
  PLXNB2-206 cDNA (coding sequence)
  variation 1: /replace=“a,c,g,t”
nagacagatgtgaacttccagggcaagaacctggacaccgtgaaggtgtggatgagtggtggaggtggccgtgggaaacccacagaggacgttcagaggcttctgcagacacagggttcctccctgcacgtgggcagtgacttgctcaagttcatggagccggtgaccatgcaggaatctgggaccttcgcctttcggaccccaaagctgtcccacgatgccaacgagacgctgcccctgcacctctacgtcaa

SEQ ID NO: 16 - protein, 84 aa, Homo sapiens
  PLXNB2-206 protein
  VARIANT 1: Ala,Cys,Asp,Glu,Phe,Gly,His,Ile,Lys,Leu,Met, Asn,Pro,Gln,Arg,Ser,Thr,Val,Trp,Tyr
XTDVNFQGKNLDTVKVWMSGGGGRGKPTEDVQRLLQTQGSSLHVGSDLLKFMEPVTMQESGTFAFRTPKLSHDANETLPLHLYV

SEQ ID NO: 17 - DNA, 483 bp, Homo sapiens
  PLXNB2-213 cDNA (coding sequence)
ggacctgacgtcacagcgggagggccggtggaagcgcgtcaacacccttatgcactacaatgtccgggatggagccaccctcatcctgtccaaggtgggggtctcccagcagccggaggacagccagcaggacctgcctggggagcgccatgccctcctggaggaggagaaccgggtgtggcacctggtgcggccgaccgacgaggtggacgagggcaagtccaagagaggcagcgtgaaagagaaggagcggacgaaggccatcaccgagatctacctgacgcggctgctctcagtcaagggcacactgcagcagtttgtggacaacttcttccagagcgtgctggcctgggcacgcggtgccacctgcagtcaagtacttcttcgacttcctggacgagcaggcagagaagcacaacatccaggatgaagacaccatccacatctggaagacgaacagcttaccgctccggttctgggtga

SEQ ID NO: 18 - protein, 160 aa, Homo sapiens
  PLXNB2-213 protein
GPDVTAGGPVEARQHPYALQCPGWSHPHPVQGGGLPAAGGQPAGPAWGAPCPPGGGEPGVAPGAADRRGGRGQVQERQREREGADEGHHRDLPDAAALSQGHTAAVCGQLLPERAGLGTRCHLQSSTSSTSWTSRQRSTTSRMKTPSTSGRRTAYRSGSG

SEQ ID NO: 19 - DNA, 1041 bp, Homo sapiens
  PLXNB2-214 cDNA (coding sequence)
atgcacacgctcttcctggagctcctggagcagtacgtggtggccaagaaccccaagctgatgctgcgcaggtctgagactgtggtggagaggatgctgtccaactggatgtccatctgcctgtaccagtacctcaaggacagtgccggggagcccctgtacaagctcttcaaggccatcaaacatcaggtggaaaagggcccggtggatgcggtacagaagaaggccaagtacactctcaacgacacggggctgctgggggatgatgtggagtacgcacccctgacggtgagcgtgatcgtgcaggacgagggagtggacgccatcccggtgaaggtcctcaactgtgacaccatctcccaggtcaaggagaagatcattgaccaggtgtaccgtgggcagccctgctcctgcggcccaggccagacagcgtggtcctggagtggcgtccgggctccacagcgcagatccgtcggacctggacctgacgtcacagcgggagggccggtggaagcgcgtcaacacccttatgcactacaatgtccgggatggagccaccctcatcctgtccaaggtgggggtctcccagcagccggaggacagccagcaggacctgcctggagcgccatgcctcctggaggaggagaaccgggtgtggcacctggtgcggccgaccgacgaggtggacgagggcaagtccaagagaggcagcgtgaaagagaaggagcggacgaaggccatcaccgagatctacctgacgcggctgctctcagtcaagggcacactgcagcagtttgtggacaacttcttccagagcgtgctggcgcctggcacgcggtgccacctgcagtcaagtacttcttcgacttcctggacgagcaggcagagaagcacaacatccaggatgaagacaccatccacatctggaagacgaacagcttaccgctccggttctgggtgaacatcctcaagaacccccacttcatctttgacgtgcatgtcacgaggtggtggacgcctcgctgtcagtcatcgcgcaga

SEQ ID NO: 20 - protein, 347 aa, Homo sapiens [sequence omitted]

(19) United States
(12) Patent Application Publication
(10) Pub. No.: US 2022/0162616 A1
(43) Pub. Date: May 26, 2022

(54) DUAL INHIBITION OF PLEXIN-B1 AND PLEXIN-B2

(71) Applicants: MAX-PLANCK-GESELLSCHAFT ZUR FÖRDERUNG DER WISSENSCHAFTEN e.V., München (DE); Philipps-Universität Marburg, Marburg (DE)

(72) Inventors: Thomas WORZFELD, Marburg (DE); Stefan OFFERMANNS, Bad Nauheim (DE)

(21) Appl. No.: 17/430,301

(22) PCT Filed: Feb. 10, 2020
(86) PCT No.: PCT/EP2020/053329

§ 371 Date: Aug. 11, 2021

(30) Foreign Application Priority Data
Feb. 11, 2019 (EP) 19156552.2

(51) Int. Cl. C12N 15/113; A61K 45/06; A61K 31/7088

(52) U.S. Cl. CPC C12N 15/1138; C12N 2320/31; A61K 31/7088; A61K 45/06

(57) ABSTRACT

The present invention relates to a composition comprising an inhibitor of plexin-B1 and an inhibitor of plexin-B2, wherein the inhibitor of plexin-B1 and/or the inhibitor of plexin-B2 is/are preferably selected from (i) an inhibitor of the nucleic acid molecule encoding plexin-B1 and/or plexin-B2 selected from a small molecule, an aptamer, a siRNA, a shRNA, a miRNA, a morpholino, a ribozyme, an antisense nucleic acid molecule, a CRISPR-Cas9-based construct, a CRISPR-Cpf1-based construct, a meganuclease, a zinc finger nuclease, and a transcription activator-like (TAL) effector (TALE) nuclease, and/or (ii) an inhibitor of the plexin-B1 and/or plexin-B2 protein selected from a small molecule, an antibody or antibody mimetic, an aptamer, wherein the antibody mimetic is preferably selected from affibodies, adnectins, anticalins, DARPins, avimers, nanofitins, affilins, Kunitz domain peptides and Fynomers®.

The present invention relates to a composition comprising an inhibitor of plexin-B1 and an inhibitor of plexin-B2, wherein the inhibitor of plexin-B1 and/or the inhibitor of plexin-B2 is/are preferably selected from (i) an inhibitor of the nucleic acid molecule encoding plexin-B1 and/or plexin-B2 selected from a small molecule, an aptamer, a siRNA, a shRNA, a miRNA, a morpholino, a ribozyme, an antisense nucleic acid molecule, a CRISPR-Cas9-based construct, a CRISPR-Cpf1-based construct, a meganuclease, a zinc finger nuclease, and a transcription activator-like (TAL) effector (TALE) nuclease, and/or (ii) an inhibitor of the plexin-B1 and/or plexin-B2 protein selected from a small molecule, an antibody or antibody mimetic, an aptamer, wherein the antibody mimetic is preferably selected from affibodies, adnectins, anticalins, DARPins, avimers, nanofitins, affilins, Kunitz domain peptides and Fynomers®.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Plexins are a family of transmembrane receptors for semaphorins, initially characterized in the context of axon guidance in the developing nervous system (Tamagnone 1990, Cell 99:71-80). Plexin-B1 has been shown to stably interact with ErbB-2 (Swiercz 2004, J Cell Biol 165:869-880). This interaction is critical for activation of the small GTPase RhoA by semaphorin ligands of Plexin-B1. The Rho family of small GTPases has been extensively studied for their role in invasion of cancer cells (Sahai 2002, Nat Rev Cancer 2: 133-42). RhoA and RhoC, in particular, are overexpressed in breast cancer and contribute to metastasis and poor outcome in breast cancer patients (Lin 2004, Breast Cancer Res Treat 84:49-60). Moreover, binding of the ligand Sema4D to its receptor plexin-B1 stimulates the kinase activity of ErbB-2 which leads to phosphorylation of plexin-B1 at two specific tyrosine residues (Swiercz 2009, Mol Cell Biol 29:6321-34). Plexin has been reported as a target protein for tumor diagnosis and therapy (US2010/119445).

Plexin-B1 and Plexin-B2 are highly homologous transmembrane receptors involved in cell-cell communication. WO 2012/107531 describes that an antagonist of plexin-B1 being capable of inhibiting the interaction between plexin-B1 and ErbB-2 can be used to treat metastatic cancer. WO 2012/135332 describes that the inhibition of plexin-B2-mediated angiogenin activity is useful for the treatment of cancer and the inhibition of angiogenesis. Moreover, EP2993185 reveals that an antibody blocking the interaction of plexin-B1 and semaphorin is useful to treat osteoporosis, multiple sclerosis and neoplastic diseases.

Although medical uses of plexins including B-type plexins are known there is an ongoing need for new treatment avenues for conditions or disease where treatment options are not available or not fully satisfactory. This need is addressed by the present application.

Accordingly the present invention relates in a first aspect to a composition comprising an inhibitor of plexin-B1 and an inhibitor of plexin-B2, wherein the inhibitor of plexin-B1 and/or the inhibitor of plexin-B2 is/are preferably selected from (i) an inhibitor of the nucleic acid molecule encoding plexin-B1 and/or plexin-B2 selected from a small molecule, an aptamer, a siRNA, a shRNA, a miRNA, a morpholino, a ribozyme, an antisense nucleic acid molecule, a CRISPR-Cas9-based construct, a CRISPR-Cpf1-based construct, a meganuclease, a zinc finger nuclease, and a transcription activator-like (TAL) effector (TALE) nuclease, and/or (ii) an inhibitor of the plexin-B1 and/or plexin-B2 protein selected from a small molecule, an antibody or antibody mimetic, an aptamer, wherein the antibody mimetic is preferably selected from affibodies, adnectins, anticalins, DARPins, avimers, nanofitins, affilins, Kunitz domain peptides and Fynomers®.

Plexins comprise a family of transmembrane receptors for semaphorins (Worzfeld & Offermanns, 2014). Based on homologies, plexins are divided into four subfamilies (A-D). The interaction of plexins with semaphorins is mediated by a highly conserved seven-blade β-propeller domain, the “sema domain” (Janssen et al., 2010; Liu et al., 2010; Nogi et al., 2010; Hota & Buck, 2012). The binding of semaphorins to plexins results in activation of a variety of signaling pathways (Jongbloets & Pasterkamp, 2014; Worzfeld & Offermanns, 2014; Verlinden et al. 2016). All plexins carry a GTPase-activating protein (GAP) domain in their intracellular part, which mediates the guanine nucleotide exchange of R-Ras, M-Ras and Rap1. The inhibition of R-Ras has been linked to the inhibition of the signal transduction of integrins. Plexins of the B-subfamily have a PDZ binding motif at their C-terminus, which mediates a stable interaction with the Rho guanine nucleotide exchange factors (RhoGEFs) LARG and PDZ-RhoGEF. An activation of plexins thereby leads to an activation of the small GTPases RhoA and RhoC (Worzfeld & Offermanns, 2014).

Plexin-B1 and Plexin-B2 share a high sequence homology. Both receptors are activated by the semaphorins 4A and 4D and carry out redundant functions in many tissues. Inhibition of Plexin-B1 can often be compensated by Plexin-B2 and vice versa (Perälä et al., 2011; Xia et al., 2015; Daviaud et al., 2016).

As used herein “plexin-B1” (also known as PLXNB1, PLEXIN 5, PLXN5, TRANSMEMBRANE PROTEIN SEP or SEP) refers to the B1 member of the plexin family. Plexin-B1 is preferably human plexin-B1. The cDNA sequence of human plexin-B1 is shown in SEQ ID NO: 1 and the amino acid sequence in SEQ ID NO: 3. Also a shorter variant of plexin-B1 can be found in the ensemble genome browser (Human GRCh38.p12). The cDNA sequence of the human plexin-B1 variant is shown in SEQ ID NO: 5 and the amino acid sequence in SEQ ID NO: 6. Hence, the human plexin-B1 has preferably a cDNA sequence of SEQ ID NO: 1 or 5 and an amino acid sequence of SEQ ID NO: 3 or 6. Among these sequences SEQ ID NOs 1 and 3 are preferred.

As used herein “plexin-B2” (also known as PLXNB2) refers to the B2 member of the plexin family. In accordance with the invention, plexin-B2 is preferably human plexin-B2. Also in accordance with the invention, the inhibitor of plexin-B1 is preferably an inhibitor of human plexin-B1 and the inhibitor of plexin-B2 is preferably an inhibitor human plexin-B2. The cDNA sequence of human plexin-B2 is shown in SEQ ID NO: 2 and the amino acid sequence in SEQ ID NO: 4. Seven shorter variants of plexin-B2 can be found in the ensemble genome browser (Human GRCh38.p12). The cDNA sequences of the human plexin-B2 variant are shown in SEQ ID NOs 7, 9, 11, 13, 15, 17 and 19, and the amino acid sequences in SEQ ID NOs 8, 10, 12, 14, 16, 18 and 20. Hence, the human plexin-B2 has preferably a cDNA sequence of SEQ ID NOs 2, 7, 9, 11, 13, 15, 17 or 19 and an amino acid sequence of SEQ ID NOs 4, 8, 10, 12, 14, 16, 18 or 20. Among these sequences SEQ ID NOs 2 and 4 are preferred.

The term “nucleic acid sequence”, “nucleic acid sequence molecule” or “nucleotide sequence”, in accordance with the present invention, includes DNA and RNA. The cDNA sequences of above SEQ ID NOs, wherein T is replaced by U are the mRNA sequences.

The inhibitor is also referred to herein below as a compound. Based on the activity and target of the compound, the compound is described as a compound inhibiting the expression of the nucleic acid molecule encoding plexin-B1 and/or plexin-B2 or as a compound inhibiting the protein plexin-B1 and/or plexin-B2. The nucleic acid molecule encoding plexin-B1 and/or plexin-B2 is also referred to herein below as nucleic acid molecule according to the invention and the protein plexin-B1 and/or plexin-B2 as protein according to the invention. Also with respect to the nucleic acid sequences, the amino acid sequences preferably correspond to both human plexin-B1 and human plexin-B2.

As will be further detailed herein below, the inhibitor of plexin-B1 and the inhibitor of plexin-B2 may be one compound or two distinct compounds. In the first case the compound is either bi-specific for plexin-B1 and plexin-B2 or is specific for both plexin-B1 and plexin-B2. In the latter case the two distinct compounds are preferably both an inhibitor of a nucleic acid molecule according to the invention or are both an inhibitor of the protein according to the invention. More preferably both compounds belong to the same class of compounds as described herein below, such as two siRNAs or two antibodies, wherein each is specific for plexin-B1 and plexin-B2, respectively.

A compound inhibiting the expression of the nucleic acid molecule and/or the protein according to the invention is in accordance with the present invention (i) is a compound lowering or preventing the transcription of the gene encoding the nucleic acid molecule and/or the protein according to the invention, or (ii) is a compound lowering or preventing the translation of the mRNA encoding the the protein according to the invention. Compounds of (i) include compounds interfering with the transcriptional machinery and/or its interaction with the promoter of said gene and/or with expression control elements remote from the promoter such as enhancers. Compounds of (ii) include compounds interfering with the translational machinery.

The compound inhibiting the expression of the nucleic acid molecule and/or the protein according to the invention specifically inhibits the expression of the nucleic acid molecule encoding plexin-B1 and/or plexin-B2 protein, and/or the plexin-B1 and/or plexin-B2 protein. With respect to the inhibitor of the plexin-B1 and/or plexin-B2 protein it is preferred that the inhibitor specifically binds to the extracellular domain of plexin-B1 and/or plexin-B2 protein. The extracellular domain of human plexin-B1 corresponds to amino acid positions 20 to 1490 of SEQ ID NO: 3. The extracellular domain of human plexin-B2 corresponds to amino acid positions 20 to 1197 of SEQ ID NO: 4. Binding to the extracellular domain avoids the necessity of an intracellular delivery of such an inhibitor.

Preferably, the transcription of the nucleic acid molecule and/or the protein according to the invention or the translation of the protein according to the invention is reduced by at least 50%, more preferred at least 75% such as at least 90% or 95%, even more preferred at least 98% and most preferred by about 100% (e.g., as compared to the same experimental set up in the absence of the compound).

A compound inhibiting the activity of the nucleic acid molecule and/or the protein according to the invention in accordance with the present invention causes said nucleic acid molecule and/or protein to perform its/their function with lowered efficiency. The compound inhibiting the activity of the nucleic acid molecule and/or the protein according to the invention specifically inhibits the activity of said nucleic acid molecule and/or protein. The compound inhibiting the activity of the nucleic acid molecule and/or the protein according to the invention may specifically inhibit the activity of said nucleic acid molecule and/or protein by interacting with the nucleic acid molecule and/or protein itself or by specifically inhibiting cells that produce said nucleic acid molecule and/or produce said protein and/or bind to said protein. Preferably, the activity of the nucleic acid molecule and/or the protein according to the invention is reduced by at least 50%, more preferred at least 75% such as at least 90% or 95%, even more preferred at least 98%, and most preferably about 100% (e.g., as compared to the same experimental set up in the absence of the compound).

The activity of the nucleic acid molecule and/or the protein according to the invention is in accordance with this invention its/their capability to induce tumorigenesis, in particular of colon cancer or to induce bone formation from osteoblasts, in particular in osteoporosis (see also the appended examples).

The efficiency of inhibition by an inhibitor can be quantified by methods comparing the level of activity in the presence of the inhibitor to that in the absence of the inhibitor. For example, the change in the amount of the nucleic acid molecule and/or the protein according to the invention formed may be used in the measurement. The efficiency of several inhibitors may be determined simultaneously in high-throughput formats. High-throughput assays, independently of being biochemical, cellular or other assays, generally may be performed in wells of microtiter plates, wherein each plate may contain 96, 384 or 1536 wells. Handling of the plates, including incubation at temperatures other than ambient temperature, and bringing into contact of test compounds with the assay mixture is preferably effected by one or more computer-controlled robotic systems including pipetting devices. In case large libraries of test compounds are to be screened and/or screening is to be effected within a short time, mixtures of, for example 10, 20, 30, 40, 50 or 100 test compounds may be added to each well. In case a well exhibits the expected activity, said mixture of test compounds may be de-convoluted to identify the one or more test compounds in said mixture giving rise to said activity.

For intracellular delivery the compounds inhibiting the expression and/or the activity of the nucleic acid molecule and/or the protein according to the invention may be formulated as vesicles, such as liposomes or exosomes. Liposomes have attracted great interest because of their specificity and the duration of action they offer from the standpoint of drug delivery. Liposomal cell-type delivery systems have been used to effectively deliver nucleic acids, such as siRNA in vivo into cells (Zimmermann et al. (2006) Nature, 441:111-114). Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are phagocytosed by macrophages and other cells in vivo. Exosomes are lipid packages which can carry a variety of different molecules including RNA (Alexander et al. (2015), Nat Commun; 6:7321). The exosomes including the molecules comprised therein can be taken up by recipient cells. Hence, exosomes are important mediators of intercellular communication and regulators of the cellular niche. Exosomes are useful for diagnostic and therapeutic purposes, since they can be used as delivery vehicles, e.g. for contrast agents or drugs.

The “small molecule” as used herein is preferably an organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. The organic molecule is preferably an aromatic molecule and more preferably a heteroaromatic molecule. In organic chemistry, the term aromaticity is used to describe a cyclic (ring-shaped), planar (flat) molecule with a ring of resonance bonds that exhibits more stability than other geometric or connective arrangements with the same set of atoms. Aromatic molecules are very stable, and do not break apart easily to react with other substances. In a heteroaromatic molecule at least one of the atoms in the aromatic ring is an atom other than carbon, e.g. N, S, or O. For all above-described organic molecules the molecular weight is preferably in the range of 200 Da to 1500 Da and more preferably in the range of 300 Da to 1000 Da.

Alternatively, the “small molecule” in accordance with the present invention may be an inorganic compound. Inorganic compounds are derived from mineral sources and include all compounds without carbon atoms (except carbon dioxide, carbon monoxide and carbonates). Preferably, the small molecule has a molecular weight of less than about 2000 Da, or less than about 1000 Da such as less than about 500 Da, and even more preferably less than about 250 Da. The size of a small molecule can be determined by methods well-known in the art, e.g., mass spectrometry. The small molecules may be designed, for example, based on the crystal structure of the target molecule, where sites presumably responsible for the biological activity can be identified and verified in in vivo assays such as in vivo high-throughput screening (HTS) assays.

The term “antibody” as used in accordance with the present invention comprises, for example, polyclonal or monoclonal antibodies. Furthermore, also derivatives or fragments thereof, which still retain the binding specificity to the target, e.g. the plexin-B1 or plexin-B2 of SEQ ID NO: 3 or 4, are comprised in the term “antibody”. Antibody fragments or derivatives comprise, inter alia, Fab or Fab′ fragments, Fd, F(ab′)₂, Fv or scFv fragments, single domain VH or V-like domains, such as VhH or V-NAR-domains, as well as multimeric formats such as minibodies, diabodies, tribodies or triplebodies, tetrabodies or chemically conjugated Fab′-multimers (see, for example, Harlow and Lane “Antibodies, A Laboratory Manual”, Cold Spring Harbor Laboratory Press, 1988; Harlow and Lane “Using Antibodies: A Laboratory Manual” Cold Spring Harbor Laboratory Press, 1999; Altshuler E P, Serebryanaya D V, Katrukha A G. 2010, Biochemistry (Mosc)., vol. 75(13), 1584; Holliger P, Hudson P J. 2005, Nat Biotechnol., vol. 23(9), 1126). The multimeric formats in particular comprise bispecific antibodies that can simultaneously bind to two different types of antigen. The first antigen can be found on the protein according to the invention. The second antigen may, for example, be a tumor marker that is specifically expressed on cancer cells or a certain type of cancer cells. Non-limiting examples of bispecific antibodies formats are Biclonics (bispecific, full length human IgG antibodies), DART (Dual-affinity Re-targeting Antibody) and BiTE (consisting of two single-chain variable fragments (scFvs) of different antibodies) molecules (Kontermann and Brinkmann (2015), Drug Discovery Today, 20(7):838-847). Further details on bispecific antibodies will provided herein below.

The term “antibody” also includes embodiments such as chimeric (human constant domain, non-human variable domain), single chain and humanised (human antibody with the exception of non-human CDRs) antibodies.

Various techniques for the production of antibodies are well known in the art and described, e.g. in Harlow and Lane (1988) and (1999) and Altshuler et al., 2010, loc. cit. Thus, polyclonal antibodies can be obtained from the blood of an animal following immunisation with an antigen in mixture with additives and adjuvants and monoclonal antibodies can be produced by any technique which provides antibodies produced by continuous cell line cultures. Examples for such techniques are described, e.g. in Harlow E and Lane D, Cold Spring Harbor Laboratory Press, 1988; Harlow E and Lane D, Using Antibodies: A Laboratory Manual, Cold Spring Harbor Laboratory Press, 1999 and include the hybridoma technique originally described by Köhler and Milstein, 1975, the trioma technique, the human B-cell hybridoma technique (see e.g. Kozbor D, 1983, Immunology Today, vol. 4, 7; Li J, et al. 2006, PNAS, vol. 103(10), 3557) and the EBV-hybridoma technique to produce human monoclonal antibodies (Cole et al., 1985, Alan R. Liss, Inc, 77-96). Furthermore, recombinant antibodies may be obtained from monoclonal antibodies or can be prepared de novo using various display methods such as phage, ribosomal, mRNA, or cell display. A suitable system for the expression of the recombinant (humanised) antibodies may be selected from, for example, bacteria, yeast, insects, mammalian cell lines or transgenic animals or plants (see, e.g., U.S. Pat. No. 6,080,560; Holliger P, Hudson P J. 2005, Nat Biotechnol., vol. 23(9), 11265). Further, techniques described for the production of single chain antibodies (see, inter alia, U.S. Pat. No. 4,946,778) can be adapted to produce single chain antibodies specific for an epitope of plexin-B1 and/or plexin-B2. Surface plasmon resonance as employed in the BIAcore system can be used to increase the efficiency of phage antibodies.

Antibodies against plexin-B1 (e.g. WO 2012/135332 and EP2993185) and plexin-B2 (e.g. WO 2012/107531) are known in the art.

As used herein, the term “antibody mimetics” refers to compounds which, like antibodies, can specifically bind antigens, such as the plexin-B1 and/or plexin-B2 of SEQ ID NO: 2 or 4 in the present case, but which are not structurally related to antibodies. Antibody mimetics are usually artificial peptides or proteins with a molar mass of about 3 to 20 kDa. For example, an antibody mimetic may be selected from the group consisting of affibodies, adnectins, anticalins, DARPins, avimers, nanofitins, affilins, Kunitz domain peptides and Fynomers®. These polypeptides are well known in the art and are described in further detail herein below.

The term “affibody”, as used herein, refers to a family of antibody mimetics which is derived from the Z-domain of staphylococcal protein A. Structurally, affibody molecules are based on a three-helix bundle domain which can also be incorporated into fusion proteins. In itself, an affibody has a molecular mass of around 6 kDa and is stable at high temperatures and under acidic or alkaline conditions. Target specificity is obtained by randomisation of 13 amino acids located in two alpha-helices involved in the binding activity of the parent protein domain (Feldwisch J, Tolmachev V.; (2012) Methods Mol Biol. 899:103-26).

The term “adnectin” (also referred to as “monobody”), as used herein, relates to a molecule based on the 10th extracellular domain of human fibronectin III (10Fn3), which adopts an Ig-like β-sandwich fold of 94 residues with 2 to 3 exposed loops, but lacks the central disulphide bridge (Gebauer and Skerra (2009) Curr Opinion in Chemical Biology 13:245-255). Adnectins with the desired target specificity, i.e. against plexin-B1 and/or plexin-B2, can be genetically engineered by introducing modifications in specific loops of the protein.

The term “anticalin”, as used herein, refers to an engineered protein derived from a lipocalin (Beste G, Schmidt F S, Stibora T, Skerra A. (1999) Proc Natl Acad Sci U S A. 96(5):1898-903; Gebauer and Skerra (2009) Curr Opinion in Chemical Biology 13:245-255). Anticalins possess an eight-stranded □-barrel which forms a highly conserved core unit among the lipocalins and naturally forms binding sites for ligands by means of four structurally variable loops at the open end. Anticalins, although not homologous to the IgG superfamily, show features that so far have been considered typical for the binding sites of antibodies: (i) high structural plasticity as a consequence of sequence variation and (ii) elevated conformational flexibility, allowing induced fit to targets with differing shape.

As used herein, the term “DARPin” refers to a designed ankyrin repeat domain (166 residues), which provides a rigid interface arising from typically three repeated β-turns. DARPins usually carry three repeats corresponding to an artificial consensus sequence, wherein six positions per repeat are randomised. Consequently, DARPins lack structural flexibility (Gebauer and Skerra, 2009).

The term “avimer”, as used herein, refers to a class of antibody mimetics which consist of two or more peptide sequences of 30 to 35 amino acids each, which are derived from A-domains of various membrane receptors and which are connected by linker peptides. Binding of target molecules occurs via the A-domain and domains with the desired binding specificity, i.e. for plexin-B1 and/or plexin-B2, can be selected, for example, by phage display techniques. The binding specificity of the different A-domains contained in an avimer may, but does not have to be identical (Weidle U H, et al., (2013), Cancer Genomics Proteomics; 10(4):155-68).

A “nanofitin” (also known as affitin) is an antibody mimetic protein that is derived from the DNA binding protein Sac7d of Sulfolobus acidocaldarius. Nanofitins usually have a molecular weight of around 7 kDa and are designed to specifically bind a target molecule, such as e.g. plexin-B1 and/or plexin-B2, by randomising the amino acids on the binding surface (Mouratou B, Béhar G, Paillard-Laurance L, Colinet S, Pecorari F., (2012) Methods Mol Biol.; 805:315-31).

The term “affilin”, as used herein, refers to antibody mimetics that are developed by using either gamma-B crystalline or ubiquitin as a scaffold and modifying amino-acids on the surface of these proteins by random mutagenesis. Selection of affilins with the desired target specificity, i.e. against plexin-B1 and/or plexin-B2, is effected, for example, by phage display or ribosome display techniques. Depending on the scaffold, affilins have a molecular weight of approximately 10 or 20 kDa. As used herein, the term affilin also refers to di- or multimerised forms of affilins (Weidle U H, et al., (2013), Cancer Genomics Proteomics; 10(4):155-68).

A “Kunitz domain peptide” is derived from the Kunitz domain of a Kunitz-type protease inhibitor such as bovine pancreatic trypsin inhibitor (BPTI), amyloid precursor protein (APP) or tissue factor pathway inhibitor (TFPI). Kunitz domains have a molecular weight of approximately 6 kDA and domains with the required target specificity, i.e. against plexin-B1 and/or plexin-B2, can be selected by display techniques such as phage display (Weidle et al., (2013), Cancer Genomics Proteomics; 10(4):155-68).

As used herein, the term “Fynomer®” refers to a non-immunoglobulin-derived binding polypeptide derived from the human Fyn SH3 domain. Fyn SH3-derived polypeptides are well-known in the art and have been described e.g. in Grabulovski et al. (2007) JBC, 282, p. 3196-3204, WO 2008/022759, Bertschinger et al (2007) Protein Eng Des Sel 20(2):57-68, Gebauer and Skerra (2009) Curr Opinion in Chemical Biology 13:245-255, or Schlatter et al. (2012), MAbs 4:4, 1-12).

Aptamers are nucleic acid molecules or peptide molecules that bind a specific target molecule. Aptamers are usually created by selecting them from a large random sequence pool, but natural aptamers also exist in riboswitches. Aptamers can be used for both basic research and clinical purposes as macromolecular drugs. Aptamers can be combined with ribozymes to self-cleave in the presence of their target molecule. These compound molecules have additional research, industrial and clinical applications (Osborne et. al. (1997), Current Opinion in Chemical Biology, 1:5-9; Stull & Szoka (1995), Pharmaceutical Research, 12, 4:465-483).

Nucleic acid aptamers are nucleic acid species that normally consist of (usually short) strands of oligonucleotides. Typically, they have been engineered through repeated rounds of in vitro selection or equivalently, SELEX (systematic evolution of ligands by exponential enrichment) to bind to various molecular targets such as small molecules, proteins, nucleic acids, and even cells, tissues and organisms.

Peptide aptamers are usually peptides or proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable peptide loop typically comprises 10 to 20 amino acids, and the scaffold may be any protein having good solubility properties. Currently, the bacterial protein Thioredoxin-A is the most commonly used scaffold protein, the variable peptide loop being inserted within the redox-active site the two cysteins lateral chains thereof being able to form a disulfide bridge. Peptide aptamer selection can be made using different systems, but the most widely used is currently the yeast two-hybrid system.

Aptamers offer the utility for biotechnological and therapeutic applications as they offer molecular recognition properties that rival those of the commonly used biomolecules, in particular antibodies. In addition to their discriminatory recognition, aptamers offer advantages over antibodies as they can be engineered completely in a test tube, are readily produced by chemical synthesis, possess desirable storage properties, and elicit little or no immunogenicity in therapeutic applications. Non-modified aptamers are cleared rapidly from the bloodstream, with a half-life of minutes to hours, mainly due to nuclease degradation and clearance from the body by the kidneys, a result of the aptamers' inherently low molecular weight. Unmodified aptamer applications currently focus on treating transient conditions such as blood clotting, or treating organs such as the eye where local delivery is possible. This rapid clearance can be an advantage in applications such as in vivo diagnostic imaging. Several modifications, such as 2′-fluorine-substituted pyrimidines, polyethylene glycol (PEG) linkage, fusion to albumin or other half life extending proteins etc. are available to scientists such that the half-life of aptamers can be increased for several days or even weeks.

The small molecule, antibody or antibody mimetic and aptamer can also be generated in the format of drug-conjugates. In this case the small molecule, antibody or antibody mimetic and aptamer in itself may not have an inhibitory effect but the inhibitory effect is only conferred by the drug. The small molecule, antibody or antibody mimetic and aptamer confer the site-specificity binding of the drug to cells producing and/or binding to the protein according to the invention. The drug is preferably capable to kill cells producing and/or binding to the protein according to the invention. Hence, by combining the targeting capabilities of moelcules binding to the protein according to the invention with the cell-killing ability of the drug, the drug conjugates become inhibitors that allow for discrimination between healthy and diseased tissue and cells. Cleavable and non-cleavable linkers to design drug conjugates are known in the art. Non-limiting examples of drugs being capable of killing cells are cytostaic drugs and radioisotopes that deliver radiation directly to the cancer cells.

It is furthermore possible to confine the binding and/or inhibitory activity of the small molecule, antibody or antibody mimetic and aptamer to certain tissues or cell-types, in particular diseased tissues or cell-types. For instance, probodies may be designed. In a probody the small molecule, antibody or antibody mimetic or aptamer is bound to a masking peptide which limits or prevents binding to the protein according to the invention and which masking peptide can be cleaved by a protease. Proteases are enzymes that digest proteins into smaller pieces by cleaving specific amino acid sequences known as substrates. In normal healthy tissue, protease activity is tightly controlled. In cancer cells, protease activity is upregulated. In healthy tissue or cells, where protease activity is regulated and minimal, the target-binding region of the probody remains masked and is thus unable to bind. On the other hand, in diseased tissue or cells, where protease activity is upregulated, the target-binding region of the probody gets unmasked and is thus able to bind and/or inhibit.

In accordance with the present invention, the term “small interfering RNA (siRNA)”, also known as short interfering RNA or silencing RNA, refers to a class of 18 to 30, preferably 19 to 25, most preferred 21 to 23 or even more preferably 21 nucleotide-long double-stranded RNA molecules that play a variety of roles in biology. Most notably, siRNA is involved in the RNA interference (RNAi) pathway where the siRNA interferes with the expression of a specific gene. In addition to their role in the RNAi pathway, siRNAs also act in RNAi-related pathways, e.g. as an antiviral mechanism or in shaping the chromatin structure of a genome.

siRNAs naturally found in nature have a well defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest. Essentially any gene for which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, it is preferred that at least one RNA strand has a 5′- and/or 3′-overhang. Preferably, one end of the double-strand has a 3′-overhang from 1 to 5 nucleotides, more preferably from 1 to 3 nucleotides and most preferably 2 nucleotides. The other end may be blunt-ended or has up to 6 nucleotides 3′-overhang. In general, any RNA molecule suitable to act as siRNA is envisioned in the present invention. The most efficient silencing was so far obtained with siRNA duplexes composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair (Elbashir et al. 2001). 2′-deoxynucleotides in the 3′ overhangs are as efficient as ribonucleotides, but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP) (Fougerolles et al. (2008), Current Opinion in Pharmacology, 8:280-285; Lu et al. (2008), Methods in Molecular Biology, vol. 437: Drug Delivery Systems—Chapter 3: Delivering Small Interfering RNA for Novel Therapeutics).

A preferred example of a siRNA is an Endoribonuclease-prepared siRNA (esiRNA). An esiRNA is a mixture of siRNA oligos resulting from cleavage of a long double-stranded RNA (dsRNA) with an endoribonuclease such as Escherichia coli RNase III or dicer. esiRNAs are an alternative concept to the usage of chemically synthesized siRNA for RNA Interference (RNAi). For the generation of esiRNAs a cDNA of a mRNA template may be amplified by PCR and tagged with two bacteriophage-promotor sequences. RNA polymerase is then used to generate long double stranded RNA that is complentary to the target-gene cDNA. This complentary RNA may be subsequently digested with RNase III from Escherichia coli to generate short overlapping fragments of siRNAs with a length between 18-25 base pairs. This complex mixture of short double stranded RNAs is similar to the mixture generated by dicer cleavage in vivo and is therefore called endoribonuclease-prepared siRNA or short esiRNA. Hence, esiRNA are a heterogeneous mixture of siRNAs that all target the same mRNA sequence. esiRNAs lead to highly specific and effective gene silencing.

A short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC).

This complex binds to and cleaves mRNAs which match the siRNA that is bound to it. si/shRNAs to be used in the present invention are preferably chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, Co., USA), Pierce Chemical (part of Perbio Science, Rockford, Ill., USA), Glen Research (Sterling, Va., USA), ChemGenes (Ashland, Mass., USA), and Cruachem (Glasgow, UK). Most conveniently, siRNAs or shRNAs are obtained from commercial RNA oligo synthesis suppliers, which sell RNA-synthesis products of different quality and costs. In general, the RNAs applicable in the present invention are conventionally synthesized and are readily provided in a quality suitable for RNAi.

Further molecules effecting RNAi include, for example, microRNAs (miRNA). Said RNA species are single-stranded RNA molecules. Endogenously present miRNA molecules regulate gene expression by binding to a complementary mRNA transcript and triggering of the degradation of said mRNA transcript through a process similar to RNA interference. Accordingly, exogenous miRNA may be employed as an inhibitor of plexin-B1 and/or plexin-B2 after introduction into the respective cells.

Morpholinos (or morpholino oligonucleotides) are synthetic nucleic acid molecules having a length of about 20 to 30 nucleotides and, typically about 25 nucleotides. Morpholinos bind to complementary sequences of target transcripts by standard nucleic acid base-pairing. They have standard nucleic acid bases which are bound to morpholine rings instead of deoxyribose rings and linked through phosphorodiamidate groups instead of phosphates (see, e.g., Summerton 1997, Antisense & Nucleic Acid Drug Development 7 (3): 187-95). Due to replacement of anionic phosphates into the uncharged phosphorodiamidate groups, ionization in the usual physiological pH range is prevented, so that morpholinos in organisms or cells are uncharged molecules. The entire backbone of a morpholino is made from these modified subunits. Unlike inhibitory small RNA molecules, morpholinos do not degrade their target RNA molecules. Rather, they sterically block binding to a target sequence within a RNA and simply getting in the way of molecules that might otherwise interact with the RNA (see, e.g., Summerton 1999, Biochimica et Biophysica Acta 1489 (1): 141-58).

A ribozyme (from ribonucleic acid enzyme, also called RNA enzyme or catalytic RNA) is an RNA molecule that catalyses a chemical reaction. Many natural ribozymes catalyse either their own cleavage or the cleavage of other RNAs, but they have also been found to catalyse the aminotransferase activity of the ribosome. Non-limiting examples of well-characterised small self-cleaving RNAs are the hammerhead, hairpin, hepatitis delta virus, and in vitro-selected lead-dependent ribozymes, whereas the group I intron is an example for larger ribozymes. The principle of catalytic self-cleavage has become well established in recent years. The hammerhead ribozymes are characterised best among the RNA molecules with ribozyme activity. Since it was shown that hammerhead structures can be integrated into heterologous RNA sequences and that ribozyme activity can thereby be transferred to these molecules, it appears that catalytic antisense sequences for almost any target sequence can be created, provided the target sequence contains a potential matching cleavage site. The basic principle of constructing hammerhead ribozymes is as follows: A region of interest of the RNA, which contains the GUC (or CUC) triplet, is selected. Two oligonucleotide strands, each usually with 6 to 8 nucleotides, are taken and the catalytic hammerhead sequence is inserted between them. The best results are usually obtained with short ribozymes and target sequences.

A recent development, also useful in accordance with the present invention, is the combination of an aptamer, recognizing a small compound, with a hammerhead ribozyme. The conformational change induced in the aptamer upon binding the target molecule can regulate the catalytic function of the ribozyme.

The term “antisense nucleic acid molecule”, as used herein, refers to a nucleic acid which is complementary to a target nucleic acid. An antisense molecule in accordance with the invention is capable of interacting with the target nucleic acid, more specifically it is capable of hybridizing with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901).

The antisense oligonucleotide is preferably a LNA-GapmeR, an Antagomir, or an antimiR.

LNA-GapmeRs or simply GapmeRs are potent antisense oligonucleotides used for highly efficient inhibition of mRNA function. GapmeRs function by RNase H dependent degradation of complementary RNA targets. They are an excellent alternative to siRNA for knockdown of mRNA. They are advantageously taken up by cell without transfection reagents. GapmeRs contain a central stretch of DNA monomers flanked by blocks of LNAs. The GapmeRs are preferably 14-16 nucleotides in length and are optionally fully phosphorothioated. The DNA gap activates the RNAse H-mediated degradation of targeted RNAs and is also suitable to target transcripts directly in the nucleus. The LNA-GapmeR technology is well established. LNA-GapmeRs are routinely designed using established algorithms. LNA-GapmeRs to a selected target are commercially available including positive and negative controls, for example, from Exiqon.

AntimiRs are oligonucleotide inhibitors that were initially designed to be complementary to a miRNA. AntimiRs against miRNAs have been used extensively as tools to gain understanding of specific miRNA functions and as potential therapeutics. As used herein, the AntimiRs are designed to be complementary to mRNA of plexin-B1 and/or plexin-B2. AntimiRs are preferably 14 to 23 nucleotides in length.

AntimiRs are preferably AntagomiRs. AntagomiRs are synthetic 2-O-methyl RNA oligonucleotides, preferably of 21 to 23 nucleotides which are preferably fully complementary to the selected target RNA. While AntagomiRs were initially designed against miRNAs they may also be designed against mRNAs. AntagomiRs are preferably synthesized with 2′-OMe modified bases (2′-hydroxyl of the ribose is replaced with a methoxy group), phosphorothioate (phosphodiester linkages are changed to phosphorothioates) on the first two and last four bases, and an addition of cholesterol motif at 3′ end through a hydroxyprolinol modified linkage. The addition of 2′-OMe and phosphorothioate modifications improve the bio-stability whereas cholesterol conjugation enhances distribution and cell permeation of the AntagomiRs.

Antisense molecules (including antisense oligonucleotides, such as LNA-GapmeR, an Antagomir, an antimiR), siRNAs and shRNAs of the present invention are preferably chemically synthesized using a conventional nucleic acid synthesizer. Suppliers of nucleic acid sequence synthesis reagents include Proligo (Hamburg, Germany), Dharmacon Research (Lafayette, Co., USA), Pierce Chemical (part of Perbio Science, Rockford, Ill., USA), Glen Research (Sterling, Va., USA), ChemGenes (Ashland, Mass., USA), and Cruachem (Glasgow, UK).

The ability of antisense molecules (including antisense oligonucleotides, such as LNA-GapmeR, an Antagomir, an antimiR), siRNA, and shRNA to potently, but reversibly, silence or inhibit a mRNA in vivo makes these molecules particularly well suited for use in the pharmaceutical composition and medical uses of the invention. Ways of administering siRNA to humans are described in De Fougerolles et al., Current Opinion in Pharmacology, 2008, 8:280-285. Such ways are also suitable for administering other small RNA molecules like antisense oligonucleotides or shRNAs. Accordingly, such pharmaceutical compositions may be administered directly formulated as a saline, via liposome based and polymer-based nanoparticle approaches, as conjugated or complexation pharmaceutical compositions, or via viral delivery systems. Direct administration comprises injection into tissue, intranasal and intratracheal administration. Liposome based and polymer-based nanoparticle approaches comprise the cationic lipid Genzyme Lipid (GL) 67, cationic liposomes, chitosan nanoparticles and cationic cell penetrating peptides (CPPs). Conjugated or complexation pharmaceutical compositions comprise PEI-complexed antisense molecules (including antisense oligonucleotides), siRNA, or shRNA. Further, viral delivery systems comprise influenza virus envelopes and virosomes.

The antisense molecules (including antisense oligonucleotides, such as LNA-GapmeR, an Antagomir, an antimiR), siRNAs, shRNAs may comprise modified nucleotides such as locked nucleic acids (LNAs). The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. Such oligomers are synthesized chemically and are commercially available. The locked ribose conformation enhances base stacking and backbone pre-organization. This significantly increases the hybridization properties (melting temperature) of oligonucleotides.

CRISPR/Cas9, as well as CRISPR-Cpf1, technologies are applicable in nearly all cells/model organisms and can be used for knock out mutations, chromosomal deletions, editing of DNA sequences and regulation of gene expression. The regulation of the gene expression can be manipulated by the use of a catalytically dead Cas9 enzyme (dCas9) that is conjugated with a transcriptional repressor to repress transcription a specific gene, here the plexin-B1 and/or plexin-B2 gene. Similarly, catalytically inactive, “dead” Cpf1 nuclease (CRISPR from Prevotella and Francisella-1) can be fused to synthetic transcriptional repressors or activators to downregulate endogenous promoters, e.g. the promoter which controls plexin-B1 and/or plexin-B2 expression. Alternatively, the DNA-binding domain of zinc finger nucleases (ZFNs) or transcription activator-like effector nucleases (TALENs) can be designed to specifically recognize the plexin-B1 and/or plexin-B2 gene or its promoter region or its 5′-UTR thereby inhibiting the expression of the plexin-B1 and/or plexin-B2 gene.

Inhibitors provided as inhibiting nucleic acid molecules that target the plexin-B1 and/or plexin-B2 gene or a regulatory molecule involved in plexin-B1 and/or plexin-B2 expression are also envisaged herein. Such molecules, which reduce or abolish the expression of plexin-B1 and/or plexin-B2 or a regulatory molecule include, without being limiting, meganucleases, zinc finger nucleases and transcription activator-like (TAL) effector (TALE) nucleases. Such methods are described in Silva et al., Curr Gene Ther. 2011;11(1):11-27; Miller et al., Nature biotechnology. 2011;29(2):143-148, and Klug, Annual review of biochemistry. 2010; 79:213-231.

Herein above a number of examples of nucleotide-based inhibitors were described, in particular a siRNA, a shRNA, and an antisense nucleic acid molecule. It is preferred that these nucleotide-based inhibitors and other nucleotide-based inhibitors of plexin-B1 and/or plexin-B2 comprise (a) a nucleic acid sequence which comprises or consists of a nucleic acid sequence being complementary to at least 12 continuous nucleotides of a nucleic acid sequence selected from SEQ ID NOs 1, 2, 5, 7, 9, 11, 13, 15, 17 and 19, preferably SEQ ID NOs 1 and 2 (or both), (b) a nucleic acid sequence which comprises or consists of a nucleic acid sequence which is at least 70% identical to the complementary strand of one or more nucleic acid sequences selected from SEQ ID NOs 1, 2, 5, 7, 9, 11, 13, 15, 17 and 19, preferably SEQ ID NOs 1 and 2, (c) a nucleic acid sequence which comprises or consists of a nucleic acid sequence according to (a) or (b), wherein the nucleic acid sequence is DNA or RNA, (d) an expression vector expressing the nucleic acid sequence as defined in any one of (a) to (c), preferably under the control of a cancer-specific promoter and/or a bone-specific promoter, or (e) a host comprising the expression vector of (d).

The nucleic acid sequence according to item (a) of this further preferred embodiment of the invention comprises or consists of a sequence which is with increasing preference complementary to at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides of one or more selected from SEQ ID NOs 1, 2, 5, 7, 9, 11, 13, 15, 17 and 19, preferably SEQ ID NOs 1 and 2 (or both). The nucleic acid sequence according to item (a) comprises or consists of antisense an oligonucleotide. Hence, the nucleic acid sequence according to item (a) reflects the above-mentioned basic principle of the antisense technology which is the use of an oligonucleotide for silencing a selected target RNA through the exquisite specificity of complementary-based pairing. Therefore, it is to be understood that the nucleic acid sequence according to item (a) is preferably in the format of a siRNA, shRNA or an antisense oligonucleotide as defined herein above. The antisense oligonucleotides are preferably LNA-GapmeRs, AntagomiRs, or antimiRs as defined herein above.

The nucleic acid sequence according to item (b) requiring at least 70% identity to the complementary strand of one or more nucleic acid sequences selected from SEQ ID NOs 1, 2, 5, 7, 9, 11, 13, 15, 17 and 19, preferably SEQ ID NOs 1 and 2 is considerably longer than the nucleic acid sequence according to item (a) which comprises an antisense oligonucleotide and comprises at least 12 continuous nucleotides of a nucleic acid sequence selected from SEQ ID NOs 1, 2, 5, 7, 9, 11, 13, 15, 17 and 19, preferably SEQ ID NOs 1 or 2 (or both). A nucleic acid sequence according to item (b) of the above preferred embodiment of the invention is capable of interacting with, more specifically hybridizing with the target plexin-B1 of plexin-B2. By formation of the hybrid the function of the respective plexin is reduced or blocked.

The sequence identity of the molecule according to item (b) in connection with a sequence selected from SEQ ID NOs 1, 2, 5, 7, 9, 11, 13, 15, 17 and 19, preferably SEQ ID NOs 1 and 2 is with increasing preference at least 75%, at least 80%, at least 85%, at least 90%, at least 92.5%, at least 95%, at least 98%, at least 99% and 100%. Preferably, the BLAST (Basic Local Alignment Search Tool) program is used for determining the sequence identity with regard to SEQ ID NOs 1, 2, 5, 7, 9, 11, 13, 15, 17 and 19, preferably SEQ ID NO: 1 and/or 2.

In the nucleic acid sequence according to item (c) the nucleotide sequences may be RNA or DNA. RNA or DNA encompasses chemically modified RNA nucleotides or DNA nucleotides. As commonly known RNA comprises the nucleotide U while DNA comprises the nucleotide T.

In accordance with items (d) and (e) of the above preferred embodiment the inhibitor may also be an expression vector or host, respectively being capable of producing a nucleic acid sequence as defined in any one of items (a) to (c).

An expression vector may be a plasmid that is used to introduce a specific transcript into a target cell. Once the expression vector is inside the cell, the protein that is encoded by the gene is produced by the cellular-transcription and translation machinery ribosomal complexes. The plasmid is in general engineered to contain regulatory sequences that act as enhancer and/or promoter regions and lead to efficient transcription of the transcript. In accordance with the present invention the expression vector preferably contains a cancer-specific promoter or and/or bone-specific promoter. Such promoter may allow a more targeted treatment of the disease site of the disease described herein below, in particular cancer or bone diseases.

Non-limiting examples of expression vectors include prokaryotic plasmid vectors, such as the pUC-series, pBluescript (Stratagene), the pET-series of expression vectors (Novagen) or pCRTOPO (Invitrogen) and vectors compatible with an expression in mammalian cells like pREP (Invitrogen), pcDNA3 (Invitrogen), pCEP4 (Invitrogen), pMC1 neo (Stratagene), and pXT1 (Stratage). Examples for plasmid vectors suitable for Pichia pastoris comprise e.g. the plasmids pAO815, pPIC9K and pPIC3.5K (all Intvitrogen). For the formulation of a pharmaceutical composition as described herein below a suitable vector is selected in accordance with good manufacturing practice. Such vectors are known in the art, for example, from Ausubel et al, Hum Gene Ther. 2011 Apr; 22(4):489-97 or Allay et al., Hum Gene Ther. May 2011; 22(5): 595-604.

A typical mammalian expression vector contains the promoter element, which mediates the initiation of transcription of mRNA, the protein coding sequence, and signals required for the termination of transcription and polyadenylation of the transcript. Moreover, elements such as origin of replication, drug resistance gene, regulators (as part of an inducible promoter) may also be included. The Iac promoter is a typical inducible promoter, useful for prokaryotic cells, which can be induced using the lactose analogue isopropylthiol-b-D-galactoside (“IPTG”). For recombinant expression and secretion, the polynucleotide of interest may be ligated between e.g. the PeIB leader signal, which directs the recombinant protein in the periplasm and the gene III in a phagemid called pHEN4 (described in Ghahroudi et al, 1997, FEBS Letters 414:521-526). Additional elements might include enhancers, Kozak sequences and intervening sequences flanked by donor and acceptor sites for RNA splicing. Highly efficient transcription can be achieved with the early and late promoters from SV40, the long terminal repeats (LTRs) from retroviruses, e.g., RSV, HTLVI, HIVI, and the early promoter of the cytomegalovirus (CMV). However, cellular elements can also be used (e.g., the human actin promoter). Suitable expression vectors for use in practicing the present invention include, for example, vectors such as pSVL and pMSG (Pharmacia, Uppsala, Sweden), pRSVcat (ATCC 37152), pSV2dhfr (ATCC 37146) and pBC12MI (ATCC 67109). Alternatively, the recombinant (poly)peptide can be expressed in stable cell lines that contain the gene construct integrated into a chromosome. The co-transfection with a selectable marker such as dhfr, gpt, neomycin, hygromycin allows the identification and isolation of the transfected cells. The transfected nucleic acid can also be amplified to express large amounts of the encoded (poly)peptide. The DHFR (dihydrofolate reductase) marker is useful to develop cell lines that carry several hundred or even several thousand copies of the gene of interest. Another useful selection marker is the enzyme glutamine synthase (GS) (Murphy et al.1991, Biochem J. 227:277-279; Bebbington et al. 1992, Bio/Technology 10:169-175). Using these markers, the mammalian cells are grown in selective medium and the cells with the highest resistance are selected. As indicated above, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. For vector modification techniques, see Sambrook and Russel (2001), Molecular Cloning: A Laboratory Manual, 3 Vol. Generally, vectors can contain one or more origins of replication (ori) and inheritance systems for cloning or expression, one or more markers for selection in the host, e.g., antibiotic resistance, and one or more expression cassettes. Suitable origins of replication (ori) include, for example, the Col E1, the SV40 viral and the M 13 origins of replication.

The sequences to be inserted into the vector can e.g. be synthesized by standard methods, or isolated from natural sources. Ligation of the coding sequences to transcriptional regulatory elements and/or to other amino acid encoding sequences can be carried out using established methods. Transcriptional regulatory elements (parts of an expression cassette) ensuring expression in prokaryotes or eukaryotic cells are well known to those skilled in the art. These elements comprise regulatory sequences ensuring the initiation of the transcription (e.g., translation initiation codon, promoters, enhancers, and/or insulators), internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers, and/or naturally-associated or heterologous promoter regions. Preferably, the nucleotide sequence as defined in item (a) of the above preferred embodiment of the invention is operatively linked to such expression control sequences allowing expression in prokaryotic or eukaryotic cells.

The host may be a prokaryotic or eukaryotic cell. A suitable eukaryotic host may be a mammalian cell, an amphibian cell, a fish cell, an insect cell, a fungal cell or a plant cell. Representative examples of bacterial cells are E. coli, Streptomyces and Salmonella typhimurium cells; of fungal cells are yeast cells; and of insect cells are Drosophila S2 and Spodoptera Sf9 cells. It is preferred that the cell is a mammalian cell such as a human cell. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells. The cell may be a part of a cell line, preferably a human cell line. Appropriate culture mediums and conditions for the above-described host cells are known in the art. The host is preferably a host cell and more preferably an isolated host cell. The host is also preferably a non-human host.

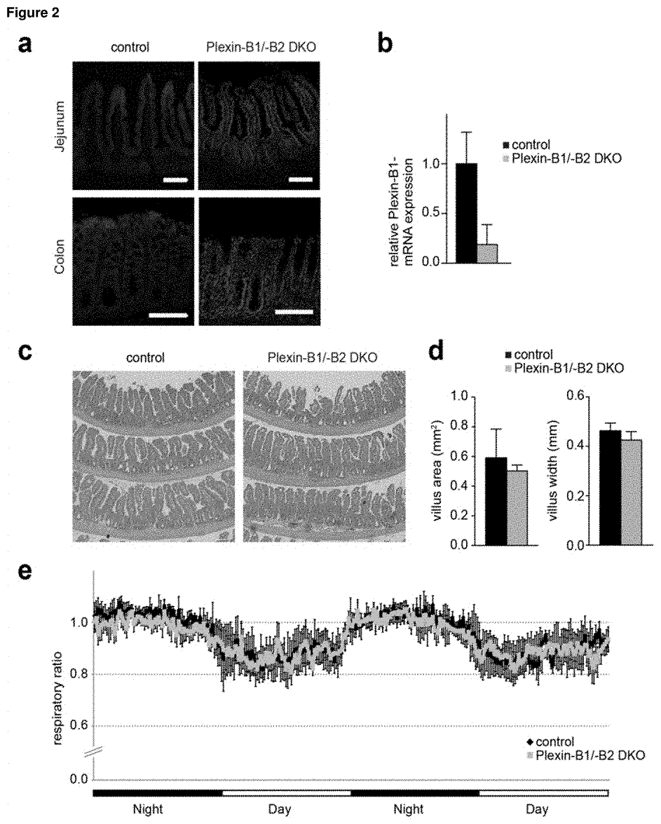

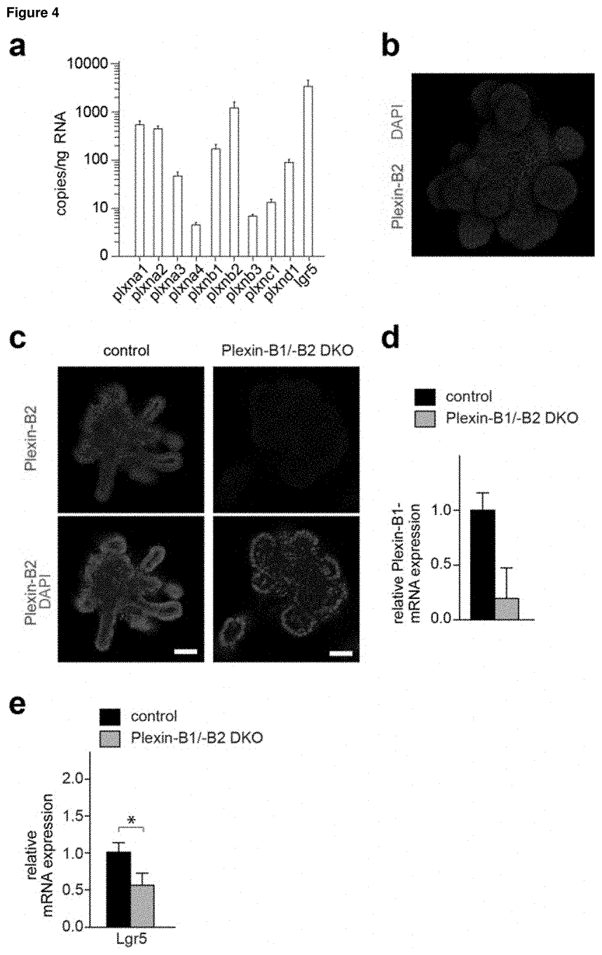

As is shown herein below in Example 1, under physiological conditions an inhibition of plexin-B1 and plexin-B2 function in the normal intestinal epithelium is compensated and stays functionally irrelevant (FIG. 2). By contrast, in tumors a dual inhibition of the function of plexin-B1 and plexin-B2 leads to a significant reduction in tumor formation (FIG. 5) and in particular a reduction which is superior as compared to the inhibition of plexin-B1 or plexin-B2. Hence, surprisingly a specific effect on tumor cells was found whereas the normal cells surrounding the tumor stayed unaffected. It was in particular unexpected that the normal cells were able to compensate the double inhibition of plexin-B1 and plexin-B2 whereas the tumor cells were not. While this is demonstrated for colon cancer, it is believed that also other cancer types can be treated in a similar manner by the double inhibition of plexin-B1 and plexin-B2.

As is furthermore shown herein below in Example 2, the inhibition of plexin-B1 and plexin-B2 advantageously promotes bone formation. Based on the data in Example 2, it can be concluded that only the double inhibition of both plexin-B1 and plexin-B2 is sufficient in order to overcome the inhibitory effect that plexin-B1 and plexin-B2 separately from each other have on the differentiation of osteoblasts into bone. The inhibition of plexin-B1 and plexin-B2 is therefore a bona fide novel treatment option for bone diseases and in particular osteoporosis. The inhibition of plexin-B1 and plexin-B2 is particularly advantageous since it not merely stops bone loss—as most currently available treatment options—but promotes bone formation. The progression of the bone disease may not only be stopped or slowed down, but an amelioration of the bone disease is expected to be achieved.

Thus, it is shown herein that the dual inhibition of plexin-B1 and plexin-B2 has surprising beneficial effects in the treatment of diseases. Therefore, the composition of the invention comprising inhibitors of plexin-B1 and plexin-B2 is highly desirable and in particular useful for the medical treatments described in more detail herein below.

In accordance with a preferred embodiment of the first aspect of the invention the composition is a pharmaceutical composition.

In the pharmaceutical composition of the invention the compounds inhibiting the expression and/or the activity of the nucleic acid molecule and/or the protein according to the invention are preferably admixed with a pharmaceutically acceptable carrier or excipient to form a pharmaceutical composition. Suitable pharmaceutically acceptable carriers or excipients as well as the formulation of pharmaceutical compositions will be discussed herein below.

Via the pharmaceutical composition of the invention the compounds inhibiting the expression and/or the activity of the nucleic acid molecule and/or the protein according to the invention can be administered to a subject at a suitable dose and/or a therapeutically effective amount. Also this will be further discussed herein below. The length of treatment needed to observe changes and the interval following treatment for responses to occur vary depending on the desired effect. The particular amounts may be determined by conventional tests which are well known to the person skilled in the art. Suitable tests are, for example, described in Tamhane and Logan (2002), “Multiple Test Procedures for Identifying the Minimum Effective and Maximum Safe Doses of a Drug”, Journal of the American statistical association, 97(457):1-9.

In accordance with the present invention, the term “pharmaceutical composition” relates to a composition for administration to a patient, preferably a human patient. The pharmaceutical composition of the invention comprises the compounds recited above. It may, optionally, comprise further molecules capable of altering the characteristics of the compounds of the invention thereby, for example, stabilizing, modulating and/or activating their function. The composition may be in solid, liquid or gaseous form and may be, inter alia, in the form of (a) powder(s), (a) tablet(s), (a) solution(s) or (an) aerosol(s). The pharmaceutical composition of the present invention may, optionally and additionally, comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, organic solvents including DMSO etc. Compositions comprising such carriers can be formulated by well known conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgement of the ordinary clinician or physician. Generally, the regimen as a regular administration of the pharmaceutical composition should be in the range of 1 μg to 5 g units per day. However, a more preferred dosage might be in the range of 0.01 mg to 100 mg, even more preferably 0.01 mg to 50 mg and most preferably 0.01 mg to 10 mg per day. Furthermore, if for example said compound is an iRNA agent, such as an siRNA, the total pharmaceutically effective amount of pharmaceutical composition administered will typically be less than about 75 mg per kg of body weight, such as for example less than about 70, 60, 50, 40, 30, 20, 10, 5, 2, 1, 0.5, 0.1, 0.05, 0.01, 0.005, 0.001, or 0.0005 mg per kg of body weight. More preferably, the amount will be less than 2000 nmol of iRNA agent (e.g., about 4.4×10¹⁶ copies) per kg of body weight, such as for example less than 1500, 750, 300, 150, 75, 15, 7.5, 1.5, 0.75, 0.15, 0.075, 0.015, 0.0075, 0.0015, 0.00075 or 0.00015 nmol of iRNA agent per kg of body weight. The length of treatment needed to observe changes and the interval following treatment for responses to occur vary depending on the desired effect.

As will be further detailed herein below, the pharmaceutical composition of the invention can be used, for example, to treat or prevent cancer and bone diseases. However, the pharmaceutical composition may also be used for the treatment or prevention of further diseases.

The present invention relates in a second aspect to an inhibitor of plexin-B1 and an inhibitor of plexin-B2 as defined in connection with the first aspect of the invention for use in the treatment or prevention of a disease.

The definitions and preferred embodiments of the first aspect of the invention apply mutatis mutandis to the second aspect of the invention as far as being applicable to this second aspect. For instance, also in the context of the second aspect the inhibitor of plexin-B1 and the inhibitor of plexin-B2 may be formulated as a pharmaceutical composition as described above.

As used herein the term “disease” encompasses any particular abnormal condition that negatively affects the structure or function of part or all of an organism including an external injury, such a bone fracture. The organism is preferably human. A disease may be caused by external factors such as pathogens or by internal dysfunctions, thereby giving raise to diseases such as cancer.

In accordance with a preferred embodiment of the second aspect of the invention the disease is cancer.

Cancer is an abnormal malignant new growth of tissue that possesses no physiological function and arises from uncontrolled usually rapid cellular proliferation.

The cancer can be selected from the group consisting of breast cancer, ovarian cancer, endometrial cancer, vaginal cancer, vulvacancer, bladder cancer, salivary gland cancer, pancreatic cancer, thyroid cancer, kidney cancer, lung cancer, cancer concerning the upper gastrointestinal tract, colon cancer, colorectal cancer, prostate cancer, squamous-cell carcinoma of the head and neck, cervical cancer, glioblastomas, malignant ascites, lymphomas and leukemias.

The cancer is preferably a solid tumor or cancer. A solid tumor or cancer is an abnormal mass of tissue that usually does not contain cysts or liquid areas by contrast to a liquid tumor.

In accordance with a more preferred embodiment of the second aspect of the invention the cancer is a colon cancer, gastrointestinal cancer, cervical cancer, ovarian cancer or bone cancer.

Colon cancer (also commonly referred to as colorectal cancer) is cancer of the large intestine (colon), which is the final part of the digestive tract. Colon cancer has a high incidence. In 2017, nearly 136,000 new cases of colorectal cancer were diagnosed in the U.S. About 1 in 20 (5%) Americans will develop colorectal cancer during their lifetime.

Gastrointestinal cancer refers to malignant conditions of the gastrointestinal tract (GI tract) and accessory organs of digestion, including the esophagus, stomach, biliary system, pancreas, small intestine, large intestine, rectum and anus. Esophageal cancer is the sixth-most-common cancer in the world, and its incidence is increasing.

Bone cancers are broken down into separate types based on the type of cell where the cancer began. The most common types and thus preferred types of bone cancer are chondrosarcoma, Ewing sarcoma and osteosarcoma. Chondrosarcoma is the second most common form of bone cancer. In this tumor, the cancerous cells produce cartilage. Chondrosarcoma usually occurs in the pelvis, legs or arms in middle-aged and older adults. Ewing sarcoma tumors most commonly arise in the pelvis, legs or arms of children and young adults. Osteosarcoma is the most common form of bone cancer. In this tumor, the cancerous cells produce bone. This variety of bone cancer occurs most often in children and young adults, in the bones of the leg or arm. In rare circumstances, osteosarcomas can arise outside of bones (extraskeletal osteosarcomas).

Cervical cancer develops in a woman's cervix (the entrance to the womb from the vagina). It mainly affects sexually active women aged between 30 and 45. This is because various strains of the human papillomavirus (HPV), a sexually transmitted infection, play a role in causing most cervical cancer.

Ovarian cancer occurs due to abnormal and uncontrolled cell growth in the ovaries. Most cases of ovarian cancer occur sporadically in subjects with little to no family history of the condition. However, approximately 10-25% of ovarian cancers are thought to be “hereditary.”

Among colon cancer, gastrointestinal cancer, cervical cancer, ovarian cancer and bone cancer colon cancer is preferred.

In accordance with a further preferred embodiment of the second aspect of the invention the disease is a bone disease.

Bone disease refers to the medical conditions which affect the bone. An example is bone cancer as described herein above. In bone diseases bones generally break easily. Different kinds of bone problems include low bone density and osteoporosis, which make the bones weak and more likely to break, osteogenesis imperfecta which makes the bones brittle, Paget's disease which makes the bones weak. Bones can also develop infections. Other bone diseases are caused by poor nutrition, genetics, or problems with the rate of bone growth or rebuilding. All these bone diseases will benefit from osteogenesis, so that the bones can become stronger again.

In accordance with a more preferred embodiment of the second aspect of the invention the bone disease is associated with bone loss.

A disease is associated with bone loss is a disease being characterized by a decrease in bone mass and/or density. Means for determining bone mass and/or density are art established. For example, Quantitative computed tomography (QCT) is a medical technique that measures bone mineral density (BMD) using a standard X-ray Computed Tomography (CT) scanner with a calibration standard to convert Hounsfield Units (HU) of the CT image to bone mineral density values. Quantitative CT scans are primarily used to evaluate bone mineral density at the lumbar spine and hip.

In accordance with an even more preferred embodiment of the second aspect of the invention the bone disease being associated with bone loss is osteoporosis or periodontosis.

Osteoporosis is a disease that weakens bones to the point where they break easily—most often, bones in the hip, backbone (spine), and wrist. Osteoporosis is called a “silent disease” because affected individuals may not notice any changes until a bone breaks. All the while, though, bones may beve losing strength for many years. It is the most common reason for a broken bone among the elderly. Osteoporosis has a prevalence of about 30% in postmenopausal women and is a major risk factor for bone fractures, reduced quality of life and immobilization.

Periodontosis (or peridontal disease) refers to the loos of aveolar bone, preferably with inflammation. It may lead to the loss of teeth.

Osteoporosis is preferred over periodontosis.

In accordance with another more preferred embodiment of the second aspect of the invention the bone disease is a bone fracture.

A bone fracture is a medical condition in which there is a partial or complete break in the continuity of the bone. As discussed above, a bone fracture may be the result or may become more likely by certain bone diseases. However, also in the absence of such diseases a bone may break due to an accident of physical strength. Thus, a bone fracture may be the result of high force impact or stress, or a minimal trauma injury as a result of certain medical conditions that weaken the bones, where the fracture may then be termed a pathologic fracture.

The present invention relates in a third aspect to a method for engineering bone comprising culturing pluri- or multipotent stem cells under conditions that mediate bone formation, wherein the conditions comprise the inhibitor of plexin-B1 and the inhibitor of plexin-B2 as defined in connection with the first aspect of the invention.

The definitions and preferred embodiments of the first and second aspect of the invention apply mutatis mutandis to the third aspect of the invention as far as being applicable to this third aspect.

Methods for culturing pluri- or multipotent stem cells under conditions that mediate bone formation are known in the art. Cellular sources of bone have been identified in bone marrow, periosteum, skeletal muscle, fat, and umbilical cord blood (Walmsey (2016), Stem Cell Rev., 12(5):524-529). Preferably bone marrow-derived mesenchymal stem cells (MSCs) are used as the stem cells. MSCs are among the longest-studied of all stem cell populations, and most osteogenic tissue engineering strategies use MSCs as the starting cell population.

The conditions that mediate bone formation generally require that the pluri- or multipotent stem cells are cultured in the presence of certain growth factors (GFs) which drive the differentiation of the pluri- or multipotent stem cells into bone cells. The main families of GFs involved in bone regeneration include fibroblast GFs (FGF), bone morphogenetic proteins (BMPs), vascular endothelial GF (VEGF), insulin-like GF (IGF) and transforming GF β (TGFβ). Particular attention has been directed toward the use of BMP-2 and BMP-7, which have been incorporated in Food and drug Administration (FDA)-approved devices for bone regeneration. The GFs used in bone tissue engineering can be classified as inflammatory GFs and cytokines, pro-osteogenic GFs and angiogenic GFs (De Witte et al. (2018), Regen Biomater.; 5(4): 197-211). Also the inhibition of plexin-B1 and plexin-B2 has a pro-osteogenic effect.

In the claimed method stem cells are preferably cultured together with biomaterial scaffolds for the constructing of bone (Willerth et al., Combining stem cells and biomaterial scaffolds for constructing tissues and cell deliver, Stembook. Cambridge Mass.: Harvard Stem Cell Institute; 2008). In this context, a scaffold is an implantable material, either natural or synthetic, that provides appropriate support to the developing bone.

The scaffold can be thought of as a delivery vehicle for a seed population that contains stem cells and as a structural support for the burgeoning tissue that forms as the cells differentiate. To encourage tissue formation, the stem cells are attached to the scaffold and transplanted immediately, sometimes in conjunction with appropriate tissue growth factors. Choice of scaffolding depends on application, as materials must be compatible with the stem cell population and the tissue that is being regenerated. Ideally, a scaffold will: 1) enable the stem cells to retain critical characteristics such as the ability to self-renew; 2) allow the cells to differentiate appropriately; 3) provide adequate support for the developing tissue; 4) conform to the mechanical specifications of the injury site; and 5) ultimately be resorbed into the body without generating toxic by-products.

Researchers have investigated a wide variety of natural and synthetic materials and composite mixtures of materials as scaffolds for bone regeneration. Natural proteins such as collagen, fibrin, silk, and polysaccharides (chains of sugar molecules) such as hyaluronic acid, and chitosan have demonstrated potential as components of bone scaffolding. These materials offer the advantages of biocompatibility, biodegradability, limited toxicity, and the ability to be molded to meet the mechanical requirements of bone.

In accordance with another more preferred embodiment of the third aspect of the invention the method is an ex vivo or in vitro method.

An ex vivo method is not practiced on the human or animal body. In vitro (meaning: in the glass) methods are performed with microorganisms, isolated cells, or biological molecules outside their normal biological context.

In accordance with another more preferred embodiment of all the aspects of the invention the inhibitor of plexin-B1 and the inhibitor of plexin-B2 are (i) two distinct compounds, (ii) a bispecific compound inhibiting plexin-B1 and plexin-B2, or (iii) a compound inhibiting both plexin-B1 and plexin-B2.

Among options (i) to (iii), options (i) and (ii) are preferred. In that regard it is further preferred that in case of (i) one of the two compounds specifically inhibits plexin-B1 whereas the other compound specifically inhibits plexin-B2. In that respect, it is understood that no cross-reactivity occurs. It is also preferred that the two inhibitory entities of the bispecific compounds of (ii) are specific for either of the two plexins.

As the two distinct compounds two of the inhibitors as described in connection with the first aspect of the invention may be used, such as a first antibody specifically binding to plexin-B1 and an antibody specifically binding to plexin-B2. Also different types/classes may be used for the differential inhibition, such as an antibody for plexin-B1 and an siRNA for plexin-B2.

A bispecific compound is a single compound with two different binding entities, one specifically binding to plexin-B1 and one specifically binding to plexin-B2. The bispecific compound may be a bispecific antibody or antibody fragment. A minimalistic bispecific antibody is composed of the antigen-binding sites of two antibodies. The single-chain Fv (scFv) format is the most commonly used derivative of the VH and VL domains representing the minimal antigen-binding site of an antibody. Due to the single-chain configuration, bispecific antibodies can be built by connecting two scFvs through a linker (connector). Rather than connecting antigen-binding sites in a tandem arrangement, single-domain antibodies, such as VH or VL domains, VHH, VNAR and nanobodies, can be used to make bispecific molecules, and, in general this approach can also be applied to scaffold proteins, an emerging class of antibody mimetics. Several examples of classes of antibody mimetics are described herein above. They may be fused to single-antigen specific antibodies or fragments thereof to generate bispecific antibody constructs. Diabodies (Db) are bivalent molecules composed of two chains, each comprising a VH and VL domain, either from the same or from different antibodies. In the diabody format, the two variable domains are connected by a short linker that is usually 5 residues, e.g., GGGGS. Because the linker length is substantially shorter than that required to allow intrachain assembly of an antigen-binding site, which would result in a scFv, two chains dimerize in a head-to-tail orientation resulting in a compact molecule with a molecular mass similar to tandem scFv (˜50 kDa). Fc-less bispecific antibodies were obtained using Fabs as the building block to which additional binding units are fused. Fabs are heterodimeric molecules composed of a light chain and a heavy chain fragment (Fd) and can thus be used to generate bivalent, bispecific molecules, but also trivalent, bi- or trispecific fusion proteins, e.g., by fusing a scFv to the C-terminus of either the light chain or Fd (bibody Fab-L-scFv, Fab-H-scFv), or to both chains (tribody, Fab-(scFv)₂) (Brinkmann and Kontermann (2017), MAbs. 2017 Feb-Mar; 9(2):182-212). Similar constructs may be produced fro antibody mimetics discussed above.

Also nucleic acid constructs may be bispecfic. A non-limiting example is an expression vector expressing a siRNA aganist plexin-B1 and second siRNA against pelxin-B2.

As discussed herein above, plexin-B1 and plexin-B2 are highly homologous transmembrane receptors. Thus, for example, a homologous epitope of human plexin-B1 and plexin-B2 protein may be used to generate an antibody or ribozyme specifically binding to human plexin-B1 and plexin-B2 but not to other proteins, including other plexins. Likewise, for example, a homologous nucleotide stretch of human plexin-B1 and plexin-B2 protein may be used to generate an siRNA, shRNA or antisense construct specifically binding to the mRNA encoding Plexin-B1 and Plexin-B2 but not to other the mRNA of other proteins, including other plexins.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The figures show.

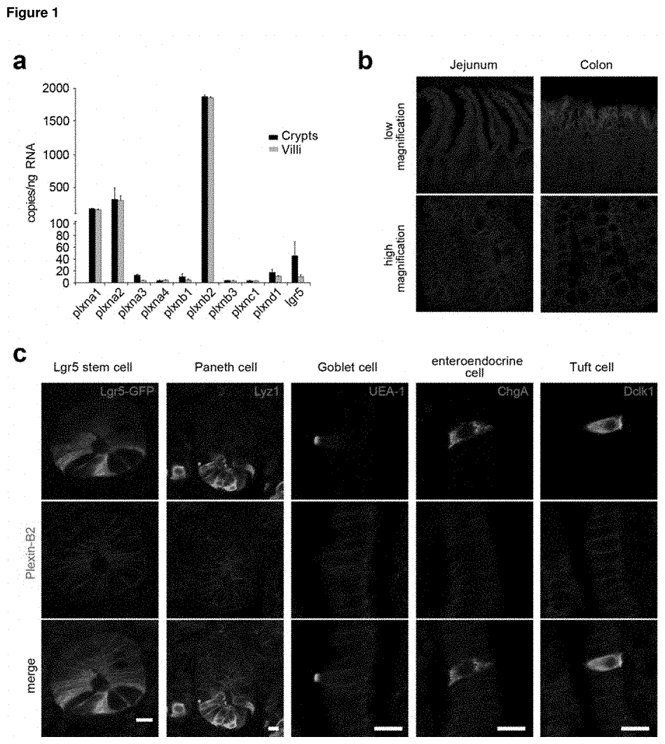

FIG. 1: Expression of Plexin-B2 in the intestinal epithelium. (a) From the mucosa of the jejunum, two fractions were isolated: one fraction contained mainly crypts, the other mainly villi. mRNA expression of plexins was determined by quantitative RT-PCR. (b) Immunstaining of Plexin-B2 (red). (c) Immunostaining for Plexin-B2 (red) on jejunum tissue sections of Lgr5 reporter mice, which express GFP under the control of the Lgr5 promoter (green; “Lgr5-GFP”; column on the very left), and double immunostainigs for Plexin-B2 (red) and cell type-specific marker proteins (green) on jejunum tissue sections of wildtype mice.

FIG. 2: Generation and analysis of intestinal epithelium-specific Plexin-B1/Plexin-B2-double-knockout mice. (a) Immunostainings for Plexin-B2 (red). (b) Quantitative RT-PCR for Plexin-B1 in the jejunum. (c) H&E-staining of tissue sections of the intestine (“Swiss Roll”). (d) Quantitative analysis of villus area and width. (e-h) Phenomaster analysis of (e) respiratory ratio, (f) food intake, (g) water intake, (h) physical activity (distance). (i) Oral glucose tolerance test. (j) 4 hours after oral administration of FITC-Dextran, FITC-Dextran was quantified in the serum by photometry. “Plexin-B1/-B2 DKO”: Plexin-B1/Plexin-B2-double-knockout.

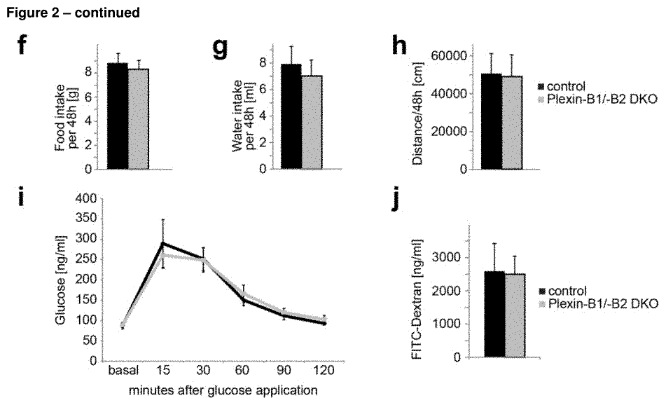

FIG. 3: Plexin-B1/-B2-double-deficient mice display a reduced number and proliferation of intestinal stem cells. (a) Quantitative RT-PCR for Lgr5 in the jejunum. (b) In-situ hybridization for Lgr5 in the mouse jejunum. (c) Quantification of the data in (b). (d) Anti-BrdU immunostaining 24 hours after BrdU injection. (e) Quantification of the distance between the crypt base and the first BrdU-positive cell along the crypt-villus axis (arrow).

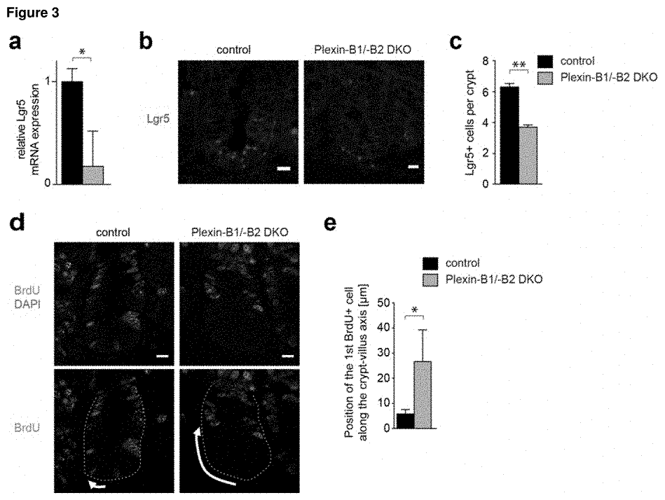

FIG. 4: Plexin-B1 and Plexin-B2 regulate Lgr5 stem cells in murine intestinal organoids. (a) Quantitative RT-PCR for plexins in intestinal murine organoids. (b) Immunostaining for Plexin-B2 (red) in intestinal organoid. (c) Immunostaining for Plexin-B2 (red) in control and Plexin-B1/Plexin-B2-double-knockout organoids. (d, e) Quantitative RT-PCR for (d) Plexin-B1 or (e) Lgr5 in control and Plexin-B1/Plexin-B2-double-knockout organoids.

FIG. 5: The inactivation of Plexin-B1 and Plexin-B2 inhibits the formation of tumors in a mouse model of colorectal tumorigenesis. Quantification of the number of tumor-bearing mice in Plexin-B1/Plexin-B2-double-deficient mice (left; PB1/PB2 DKO) and in Plexin-B2-single-deficient mice (right; PB2 SKO).

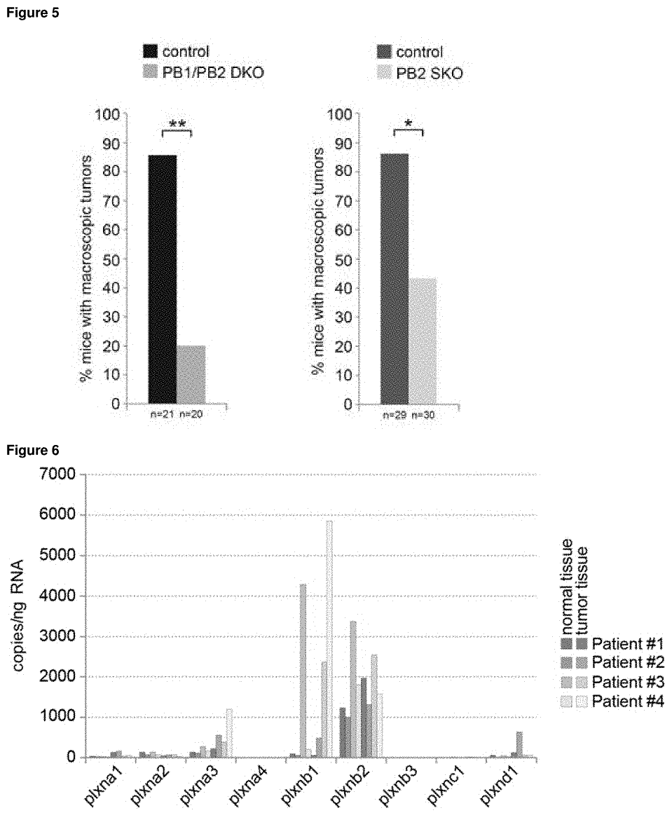

FIG. 6: Expression of plexins in human intestinal tissue. (a) Quantitative RT-PCR for plexins in normal human colon epithelium and in colon adenocarcinomas.

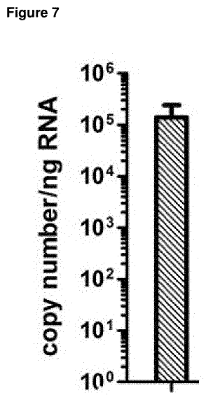

FIG. 7. Plexin-B2 is expressed by primary murine osteoblasts. Quantitative PCR for Plexin-B2 mRNA in primary murine osteoblasts.

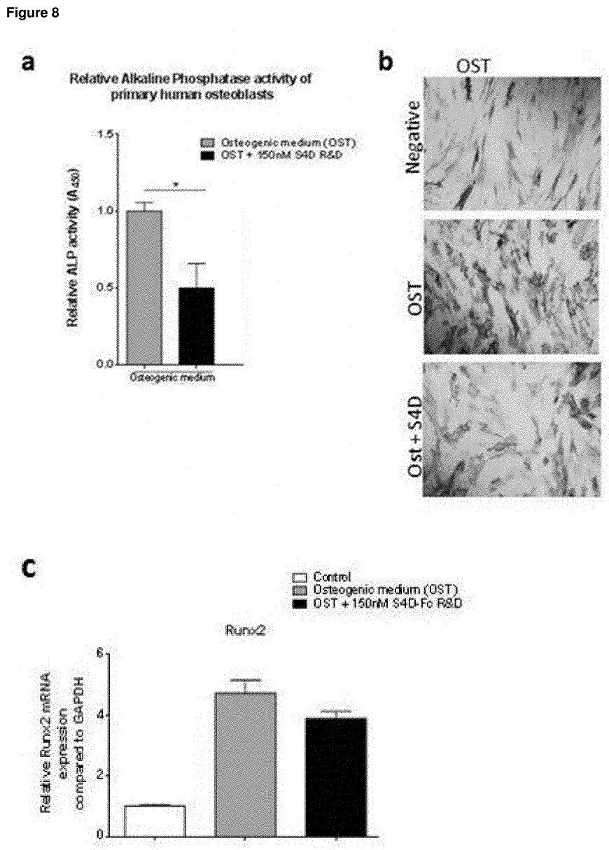

FIG. 8. Activation of Plexin-B1/Plexin-B2 inhibits differentiation of osteoblasts. (a) Application of Sema4D (150 nM) to primary human osteoblasts in vitro inhibits differentiation of osteoblasts (measured by activity of alkaline phosphatase, ALP). (b) Representative pictures of the quantification in (a). (c) Also the expression of Runx2, a marker for osteoblast differentiation, is inhibited by application of Sema4D.

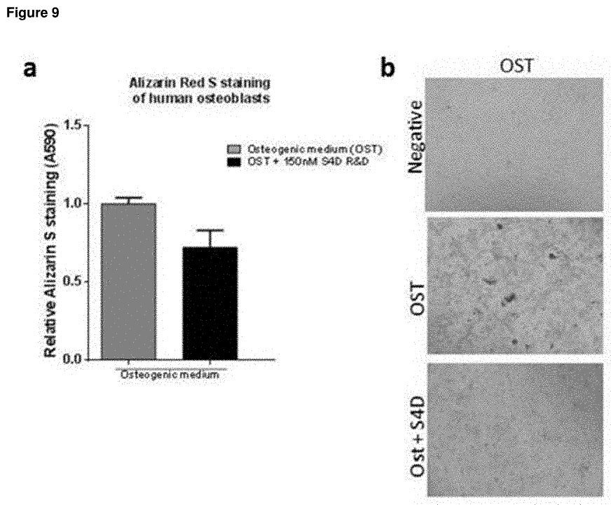

FIG. 9. Activation of Plexin-B1/Plexin-B2 inhibits mineralization of osteoblasts. (a) Application of Sema4D (150 nM) to primary human osteoblasts in vitro inhibits mineralization of osteoblasts (measured by Alizarin Red S staining). (b) Representative pictures of the quantification in (a).

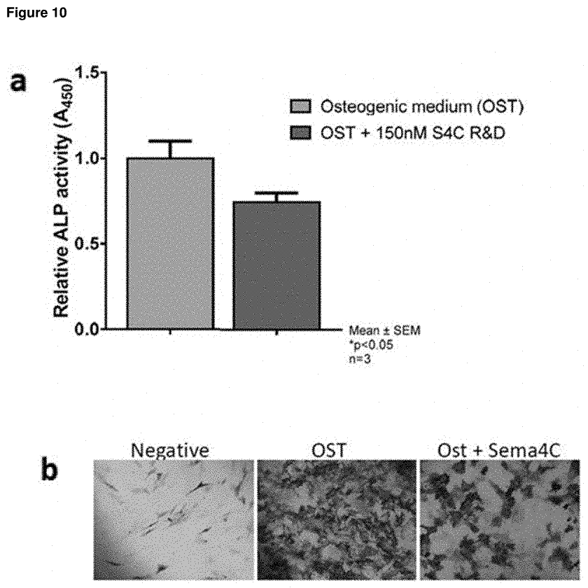

FIG. 10. Activation of Plexin-B2 inhibits osteoblast function. (a) Application of Sema4C (150 nM) to primary murine osteoblasts in vitro inhibits differentiation of osteoblasts (measured by the activity of alkaline phosphatase, ALP). (b) Representative pictures of the quantification in (a).

The examples illustrate the invention.

### EXAMPLE 1—PLEXIN-B1 AND PLEXIN-B2 IN COLORECTAL CANCER

Colorectal cancer is one of the most prevalent cancers. Intestinal stem cells, which express the marker gene Lgr5, are centrally involved in the pathogenesis of colorectal cancer. In genetic mouse models (Kozar et al., 2013; Schepers et al., 2012) as well by employing human intestinal organoids from colorectal cancer patients (Cortina et al., 2017; Shimokawa et al., 2017) it was shown that neoplasias of the intestine are hierarchially organized, and that Lg5-positive cells act as cancer stem cells. Ablation of Lgr5 cells in murine or human colorectal cancer organoids leads to a growth arrest (Shimokawa et al., 2017, de Sousa e Melo et al., 2017). However, a therapeutic approach for colorectal cancer targeting cancer stem cells is currently not yet available.

Systematic mRNA expression analyses show that Plexin-B2 is the most highly expressed plexin of the intestinal mucosa (FIG. 1a). Also on the protein level, a strong expression of Plexin-B2 in the epithelium of the small and large intestine could be observed (FIG. 1b). Co-immunostainings for Plexin-B2 and cell type-specific marker proteins, as well as immunostainings for Plexin-B2 on tissue of Lgr5 reporter mice revealed that Plexin-B2 is expressed in Lgr5-positive stem cells (FIG. 1c). Plexin-B1 is enriched in Lgr5-positive intestinal stem cells (Munoz et al., 2012).

Mice with intestinal epithelium-specific inactivation of the genes encoding Plexin-B1 und Plexin-B2 showed no significant morphological, metabolic or other functional abnormalities under physiological conditions (FIG. 2).

Plexin-B1/Plexin-B2-double-deficient mice show a reduced mRNA expression of the stem cell-specific marker gene Lgr5 (FIG. 3a), a reduction of the number of Lgr5-positive stem cells (FIG. 3b und c) and a reduced proliferation of Lgr5 stem cells (FIG. 3d und e).

Under physiological conditions, Lgr5-positive stem cells are not essential for homeostasis of the intestinal epithelium: an ablation of Lgr5-positive stem cells can be compensated for by other (reserve) stem cell populations or by dedifferentiation of differentiated intestinal epithelial cells (Tetteh et al., 2016; Tian et al., 2011). For the progression of colorectal cancer, however, Lgr5-positive stem cells are essential (see above).

In intestinal organoids, which recapitulate the physiology and the functionality of the intestinal epithelium in vivo to a large extent (Sato et al., 2009), our data show that Plexin-B2—exactly like in the intestinal epithelium in vivo—is the most highly expressed plexin (FIG. 4a und b). Plexin-B1 is also expressed in intestinal organoids (FIG. 4a). Consistent with the findings in the intestinal epithelium of Plexin-B1/-B2-knockout mice in vivo, Plexin-B1/-Plexin-B2-double-deficient organoids (FIG. 4c und d), have a reduced expression of Lgr5 (FIG. 4e). These results show that Plexin-B1 and Plexin-B2 exert an epithel-intrinsic function independently of non-epithelial intestinal cells.

In a genetic mouse model of colorectal cancer (Apc^{min} mutation), Plexin-B1/Plexin-B2-double knockout mice bear tumors much less frequently than respective control mice (FIG. 5, left). The reduction of tumor formation is less strong in Plexin-B2 single-deficient mice as compared to Plexin-B1/Plexin-B2-double-deficient mice (FIG. 5, right).

Also in the human intestine, both in normal as well as in tumor tissue, Plexin-B1 and Plexin-B2 are the most highly expressed plexins (FIG. 6).

Thus, Plexin-B1 and Plexin-B2 play a central role for the function of Lgr5-positive stem cells. Under physiological conditions, an inhibition of Plexin-B1 and Plexin-B2 function in the intestinal epithelium is compensated and stays functionally irrelevant. In tumors, however, a dual inhibition of the function of Plexin-B1 and Plexin-B2 leads to a significant reduction in tumor formation. In contrast to all other available therapeutic approaches, this strategy targets the function of cancer stem cells.

### EXAMPLE 2—PLEXIN-B1 AND PLEXIN-B2 IN OSTEOPEROSIS

Osteoporosis has a prevalence of around 30% among postmenopausal women, and represents a major risk factor for bone fractures, reduced life quality and immobilization. The currently approved drugs comprise estrogens and selective estrogen receptor modulators, bisphosphonates, the anti-RANKL antibody Denosumab, Strontium and parathyroid hormone (including its analogue Teriparatide). Despite of progress in the pharmacotherapy of osteoporosis, treatment options are still clearly insufficient and leave room for novel approaches with significant additional benefit.

Plexin-B1 (Dacquin et al., 2011; Negishi-Koga et al., 2011) and Plexin-B2 (FIG. 7) are both highly expressed on osteoblasts. A ligand for Plexin-B1 and Plexin-B2, Sema4D, is strongly expressed on osteoclasts (Dacquin et al., 2011; Negishi-Koga et al., 2011). Binding of Sema4D to Plexin-B1 results in the activation of the small GTPase RhoA and an inhibition of bone formation. Plexin-B1-deficient and Sema4D-deficient mice, and mice that express dominant-negative RhoA specifically in osteoblasts, display increased bone formation (Dacquin et al., 2011; Negishi-Koga et al., 2011). An anti-Sema4D antibody, which blocks binding of Sema4D to Plexin-B1, prevents bone loss in a mouse model for postmenopausal osteoporosis (Negishi-Koga et al., 2011).

Sema4D, which binds and activates both Plexin-B1 as well as Plexin-B2, inhibits the differentiation (FIG. 8) and mineralization (FIG. 9) of primary human osteoblasts in vitro.

Likewise, incubation of primary murine osteoblasts with Semaphorin 4C (Sema4C), a Plexin-B2-selective ligand, inhibits the differentiation of osteoblasts (FIG. 10).

The dual inhibition of Plexin-B1 and Plexin-B2, e.g. by inhibition of the binding of Plexin-B1/Plexin-B2 ligands, blocks the inhibitory effect of Plexin-B1 and Plexin-B2 on osteoblast differentiation, thereby counteracting osteoporosis development. Importantly and in contrast to most approved anti-osteoporotic drugs, this therapeutic principle relies on the enhancement of bone formation rather than on an inhibition of bone resorption.

### REFERENCES

Cortina, C., Turon, G., Stork, D., Hernando-Momblona, X., Sevillano, M., Aguilera, M., Tosi, S., Merlos-Suárez, A., Stephan-Otto Attolini, C., Sancho, E., Batlle, E. A genome editing approach to study cancer stem cells in human tumors. EMBO Molecular Medicine 9:869-879 (2017)

Dacquin, R., Domenget, C., Kumanogoh, A., Kikutani, H., Jurdic, P., and Machuca-Gayet, I. 2011. Control of bone resorption by semaphorin 4D is dependent on ovarian function. PLoS One 6:e26627.

Daviaud, N., Chen, K., Huang, Y., Friedel, R. H., Zou, H. Impaired cortical neurogenesis in plexin-B1 and -B2 double deletion mutant. Developmental Neurobiology 76:882-899 (2016)

de Sousa e Melo, F., Kurtova, A. V., Harnoss, J. M., Kljavin, N., Hoeck, J. D., Hung, J., Anderson, J. E., Storm, E. E., Modrusan, Z., Koeppen, H., Dijkgraaf, G. J., Piskol, R., de Sauvage, F. J. A distinct role for Lgr5+ stem cells in primary and metastatic colon cancer. Nature 543:676-680 (2017)

Hota P K, and Buck M. Plexin structures are coming: opportunities for multilevel investigations of semaphorin guidance receptors, their cell signaling mechanisms, and functions. Cell Mol Life Sci. 2012;69(22):3765-805.

Janssen B J, Robinson R A, Perez-Branguli F, Bell C H, Mitchell K J, Siebold C, et al. Structural basis of semaphorin-plexin signalling. Nature. 2010;467(7319):1118-22.

Jongbloets B C, Pasterkamp R J. Semaphorin signaling during development. Development. 2014; 141(17):3292-7.

Kozar, S., Morrissey, E., Nicholson, A. M., van der Heijden, M., Zecchini, H. I., Kemp, R., Tavaré, S., Vermeulen, L., Winton, D. J. Continuous clonal labeling reveals small numbers of functional stem cells in intestinal crypts and adenomas. Cell Stem Cell 13:626-633 (2013)

Liu H, Juo Z S, Shim A H, Focia P J, Chen X, Garcia K C, et al. Structural basis of semaphorin-plexin recognition and viral mimicry from Sema7A and A39R complexes with PlexinC1. Cell. 2010;142(5):749-61.

Muñoz, J., Stange, D. E., Schepers, A. G., van de Wetering, M., Koo, B. K., Itzkovitz, S., Volckmann, R., Kung, K. S., Koster, J., Radulescu, S., Myant, K., Versteeg, R., Sansom, O. J., van Es, J. H., Barker, N., van Oudenaarden, A., Mohammed, S., Heck, A. J., Clevers, H. The Lgr5 intestinal stem cell signature: robust expression of proposed quiescent ‘+4’ cell markers. EMBO Journal 31:3079-3091 (2012)

Negishi-Koga, T., Shinohara, M., Komatsu, N., Bito, H., Kodama, T., Friedel, R. H., and Takayanagi, H. 2011. Suppression of bone formation by osteoclastic expression of semaphorin 4D. Nat Med 17:1473-1480.

Nogi T, Yasui N, Mihara E, Matsunaga Y, Noda M, Yamashita N, et al. Structural basis for semaphorin signalling through the plexin receptor. Nature. 2010;467(7319):1123-7.

Perälä, N., Jakobson, M., Ola, R., Fazzari, P., Penachioni, J. Y., Nymark, M., Tanninen, T., Immonen, T., Tamagnone, L., Sariola, H. Sema4C-Plexin B2 signalling modulates ureteric branching in developing kidney. Differentiation 81:81-91 (2011)

Sato, T., Vries, R. G., Snippert, H. J., van de Wetering, M., Barker, N., Stange, D. E., van Es, J. H., Abo, A., Kujala, P., Peters, P. J., Clevers, H. Single Lgr5 stem cells build crypt-villus structures in vitro without a mesenchymal niche. Nature 2009 459:262-265 (2009)

Schepers, A. G., Snippert, H. J., Stange, D. E., van den Born, M., van Es, J. H., van de Wetering, M., Clevers, H. Lineage tracing reveals Lgr5+ stem cell activity in mouse intestinal adenomas. Science 337:730-735 (2012)

Shimokawa, M., Ohta, Y., Nishikori, S., Matano, M., Takano, A., Fujii, M., Date, S., Sugimoto, S., Kanai, T., Sato, T. Visualization and targeting of LGR5+ human colon cancer stem cells. Nature 545:187-192 (2017)

Tetteh, P. W., Basak, O., Farin, H. F., Wiebrands, K., Kretzschmar, K., Begthel, H., van den Born, M., Korving, J., de Sauvage, F., van Es, J. H., van Oudenaarden, A., Clevers, H. Replacement of Lost Lgr5-Positive Stem Cells through Plasticity of Their Enterocyte-Lineage Daughters. Cell Stem Cell 18:203-213 (2016)

Tian, H., Biehs, B., Warming, S., Leong, K. G., Rangell, L., Klein, O. D., de Sauvage, F. J. A reserve stem cell population in small intestine renders Lgr5-positive cells dispensable. Nature 478:255-259 (2011)

Verlinden L, Vanderschueren D, Verstuyf A. Semaphorin signaling in bone. Mol Cell Endocrinol. 2016; 432:66-74.

Worzfeld T, Offermanns, S. Semaphorins and plexins as therapeutic targets. Nat Rev Drug Discov. 2014;13:603-621.

Xia, J., Swiercz, J. M., Bañón-Rodríguez, I., Matković, I., Federico, G., Sun, T., Franz, T., Brakebusch, C. H., Kumanogoh, A., Friedel, R. H., Martín-Belmonte, F., Gröne, H. J., Offermanns, S., Worzfeld, T. Semaphorin-Plexin Signaling Controls Mitotic Spindle Orientation during Epithelial Morphogenesis and Repair. Developmental Cell 33, 299-313 (2015)

1. A method of treating or preventing a disease, comprising administering an inhibitor of plexin-B 1 and an inhibitor of plexin-B2, wherein the inhibitor of plexin-B 1 and the inhibitor of plexin-B2 are selected from:
(i) an inhibitor of the nucleic acid molecule encoding plexin-B 1 and/or plexin-B2 selected from an aptamer, a siRNA, a shRNA, a miRNA, a morpholino, a ribozyme, an antisense nucleic acid molecule, a CRISPR-Cas9-based construct, a CRISPR-Cpf1-based construct, a meganuclease, a zinc finger nuclease, and a transcription activator-like (TAL) effector (TALE) nuclease, and/or
(ii) an inhibitor of the plexin-B 1 and/or plexin-B2 protein selected from an antibody or antibody mimetic, an aptamer, wherein the antibody mimetic is selected from affibodies, adnectins, anticalins, DARPins, avimers, nanofitins, affilins, Kunitz domain peptides and Fynomers®, and
wherein the inhibitors specifically inhibit the expression of the nucleic acid molecule encoding plexin-B 1 and/or plexin-B2 protein, and/or the plexin-B 1 and/or plexin-B2 protein.

2. The method of claim 1, wherein the disease is cancer.

3. The method of claim 2, wherein the cancer is a colon cancer, gastrointestinal cancer, cervical cancer, ovarian cancer or bone cancer.

4. The method of claim 1, wherein the disease is bone disease.

5. The method of claim 4, wherein the bone disease is associated with bone loss.

6. The method of claim 4, wherein the bone disease associated with bone loss is osteoporosis or periodontosis.

7. The method of claim 4, wherein the bone disease is a bone fracture.

8. A method for engineering bone comprising culturing pluri- or multipotent stem cells under conditions that mediate bone formation, wherein the conditions comprise the inhibitor of plexin-B 1 and the inhibitor of plexin-B2 wherein the inhibitor of plexin-B1 and the inhibitor of plexin-B2 are selected from:
(i) an inhibitor of the nucleic acid molecule encoding plexin-B 1 and/or plexin-B2 selected from an aptamer, a siRNA, a shRNA, a miRNA, a morpholino, a ribozyme, an antisense nucleic acid molecule, a CRISPR-Cas9-based construct, a CRISPR-Cpf1-based construct, a meganuclease, a zinc finger nuclease, and a transcription activator-like (TAL) effector (TALE) nuclease, and/or
(ii) an inhibitor of the plexin-B 1 and/or plexin-B2 protein selected from an antibody or antibody mimetic, an aptamer, wherein the antibody mimetic is selected from affibodies, adnectins, anticalins, DARPins, avimers, nanofitins, affilins, Kunitz domain peptides and Fynomers®, and
wherein the inhibitors specifically inhibit the expression of the nucleic acid molecule encoding plexin-B 1 and/or plexin-B2 protein, and/or the plexin-B 1 and/or plexin-B2 protein.

9. The method of claim 8, wherein the method is an ex vivo or in vitro method.

10. A composition for treating or preventing a disease comprising an inhibitor of plexin-B 1 and the inhibitor of plexin-B2, wherein the inhibitor of plexin-B 1 and the inhibitor of plexin-B2 are selected from:
(i) an inhibitor of the nucleic acid molecule encoding plexin-B 1 and/or plexin-B2 selected from an aptamer, a siRNA, a shRNA, a miRNA, a morpholino, a ribozyme, an antisense nucleic acid molecule, a CRISPR-Cas9-based construct, a CRISPR-Cpf1-based construct, a meganuclease, a zinc finger nuclease, and a transcription activator-like (TAL) effector (TALE) nuclease, and/or
(ii) an inhibitor of the plexin-B 1 and/or plexin-B2 protein selected from an antibody or antibody mimetic, an aptamer, wherein the antibody mimetic is selected from affibodies, adnectins, anticalins, DARPins, avimers, nanofitins, affilins, Kunitz domain peptides and Fynomers®,
wherein the inhibitors specifically inhibit the expression of the nucleic acid molecule encoding plexin-B 1 and/or plexin-B2 protein, and/or the plexin-B 1 and/or plexin-B2 protein, and wherein the inhibitor of plexin-B 1 and the inhibitor of plexin-B2 are (i) two distinct compounds, (ii) a bispecific compound inhibiting Plexin-B 1 and Plexin-B2, or (iii) a compound inhibiting both Plexin-B 1 and Plexin-B2.

11. The method of claim 8, wherein the inhibitor of plexin-B 1 and the inhibitor of plexin-B2 are (i) two distinct compounds, (ii) a bispecific compound inhibiting Plexin-B1 and Plexin-B2, or (iii) a compound inhibiting both Plexin-B1 and Plexin-B2.
